(19) United States
(12) Patent Application Publication
(10) Pub. No.: US 2006/0211644 A1
(43) Pub. Date: Sep. 21, 2006

(54) Immunostimulatory oligonucleotides

(75) Inventors: Arthur M. Krieg, Wellesley, MA (US); Ulrike Samulowitz, Lagenfeld (DE); Jorg Vollmer, Duesseldorf (DE)

Correspondence Address: WOLF GREENFIELD & SACKS, PC, FEDERAL RESERVE PLAZA, 600 ATLANTIC AVENUE, BOSTON, MA, 02210-2206, US

(73) Assignees: Coley Pharmaceutical Group, Inc., Wellesley, MA (US); Coley Pharmaceutical GmbH, Langenfeld (DE)

(21) Appl. No.: 11/361,313

(22) Filed: Feb. 24, 2006

Related U.S. Application Data
(60) Provisional application No. 60/655,931, filed on Feb. 24, 2005.

(51) Int. Cl. A61K 48/00; C07H 21/02

(52) U.S. Cl. 514/44; 536/23.1

(57) ABSTRACT

The invention relates to a class of short CpG immunostimulatory oligonucleotides that are useful for stimulating an immune response. Preferably the short oligonucleotides are soft or semi-soft oligonucleotides.

### RELATED APPLICATION

This application claims priority to U.S. Provisional Application No. 60/655,931, filed Feb. 24, 2005, the entire contents of which are hereby incorporated by reference.

### FIELD OF THE INVENTION

The present invention relates generally to short immunostimulatory oligonucleotides, as well as immunostimulatory oligonucleotides with reduced renal inflammatory effects, compositions thereof and methods of using the immunostimulatory oligonucleotides.

### BACKGROUND OF THE INVENTION

Bacterial DNA has immune stimulatory effects to activate B cells and natural killer cells, but vertebrate DNA does not (Tokunaga, T., et al., 1988. Jpn. J Cancer Res. 79:682-686; Tokunaga, T., et al., 1984, JNCI72:955-962; Messina, J. P., et al., 1991, J. Immunol. 147:1759-1764; and reviewed in Krieg, 1998, In: Applied Oligonucleotide Technology, C. A. Stein and A. M. Krieg, (Eds.), John Wiley and Sons, Inc., New York, N.Y., pp. 431-448). It is now understood that these immune stimulatory effects of bacterial DNA are a result of the presence of unmethylated CpG dinucleotides in particular base contexts (CpG motifs), which are common in bacterial DNA, but methylated and underrepresented in vertebrate DNA (Krieg et al, 1995 Nature 374:546-549; Krieg, 1999 Biochim. Biophys. Acta 93321:1-10). The immune stimulatory effects of bacterial DNA can be mimicked with synthetic oligodeoxynucleotides (ODN) containing these CpG motifs. Such CpG ODN have highly stimulatory effects on human and murine leukocytes, inducing B cell proliferation; cytokine and immunoglobulin secretion; natural killer (NK) cell lytic activity and IFN-γ secretion; and activation of dendritic cells (DCs) and other antigen presenting cells to express costimulatory molecules and secrete cytokines, especially the Th1-like cytokines that are important in promoting the development of Th1-like T cell responses. These immune stimulatory effects of native phosphodiester backbone CpG ODN are highly CpG specific in that the effects are dramatically reduced if the CpG motif is methylated, changed to a GpC, or otherwise eliminated or altered (Krieg et al, 1995 Nature 374:546-549; Hartmann et al, 1999 Proc. Natl. Acad. Sci USA 96:9305-10).

In early studies, it was thought that the immune stimulatory CpG motif followed the formula purine-purine-CpG-pyrimidine-pyrimidine (Krieg et al, 1995 Nature 374:546-549; Pisetsky, 1996 J. Immunol. 156:421-423; Hacker et al., 1998 EMBO J. 17:6230-6240; Lipford et al, 1998 Trends in Microbiol. 6:496-500). However, it is now clear that mouse lymphocytes respond quite well to phosphodiester CpG motifs that do not follow this “formula” (Yi et al., 1998 J. Immunol. 160:5898-5906) and the same is true of human B cells and dendritic cells (Hartmann et al, 1999 Proc. Natl. Acad. Sci USA 96:9305-10; Liang, 1996 J. Clin. Invest. 98:1119-1129).

Several different classes of CpG nucleic acids have recently been described. One class is potent for activating B cells but is relatively weak in inducing IFN-α and NK cell activation; this class has been termed the B class. The B class CpG nucleic acids typically are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068. Another class of CpG nucleic acids activates B cells and NK cells and induces IFN-α; this class has been termed the C-class. The C-class CpG nucleic acids, as first characterized, typically are fully stabilized, include a B class-type sequence and a GC-rich palindrome or near-palindrome. This class has been described in co-pending U.S. provisional patent application 60/313,273, filed Aug. 17, 2001 and US 10/224,523 filed on Aug. 19, 2002 and related PCT patent application PCT/US02/26468 published under International Publication Number WO 03/015711.

### SUMMARY OF THE INVENTION

It has been surprisingly discovered that immunostimulatory properties of the B-class and C-class CpG oligonucleotides and other stabilized immunostimulatory oligonucleotides can be maintained or even improved by the selective inclusion of one or more non-stabilized linkages between certain nucleotides. The non-stabilized linkages are preferably natural linkages, i.e., phosphodiester linkages or phosphodiester-like linkages. A non-stabilized linkage will typically, but not necessarily, be relatively susceptible to nuclease digestion. The immunostimulatory oligonucleotides of the instant invention include at least one non-stabilized linkage situated between a 5′ nucleotide comprising a pyrimidine (Y) base, preferably a C, and an adjacent 3′ nucleotide comprising a purine (Z) base, preferably a guanine (G), wherein both the 5′ Y and the 3′ Z are internal nucleotides. It has also been discovered that oligonucleotides of shorter lengths are effective in promoting an immune response.

In some aspects the invention is an oligonucleotide of 3 to 24 nucleotides in length comprising at least one YZ dinucleotide with a phosphodiester or phosphodiester-like internucleotide linkage, and at least 4 T nucleotides. Y is a nucleotide comprising a pyrimidine or modified pyrimidine base. Z is a nucleotide comprising a guanine or modified guanine. The oligonucleotide also includes at least one stabilized internucleotide linkage. In one embodiment the oligonucleotide includes a TTTT motif.

In other embodiments the oligonucleotide has only one YZ dinucleotide. Optionally the oligonucleotide is G*T*C_G*T*T*T*T*G*A*C (SEQ ID NO.: 16) or G*T*T*T*T*G*A*C_G*T*T*T*T*G*T*C (SEQ ID NO.: 11). * refers to the presence of a stabilized internucleotide linkage. _refers to the presence of a phosphodiester internucleotide linkage.

In other embodiments the oligonucleotide has only two YZ dinucleotides. Optionally the oligonucleotide is T*C_G*T*T*T*T*G*A*C_G*T*T (SEQ ID NO.: 3), T*C_G*T*C_G*T*T*T*T*G*A*C (SEQ ID NO.: 10), G*T*T*T*T*G*A*C_G*T*T*T*T*G*T*C₁₃ G*T*T (SEQ ID NO.: 12), G*T*C_G*T*T*T*T*G*A*C_G*T*T (SEQ ID NO.: 13), T*C_G*T*T*T*T*G*A*C_G*T*T*T*T*G*T*C (SEQ ID NO.: 14), or G*T*C_G*T*T*T*T*G*A*C_G*T*T*T*T*G*T*C (SEQ ID NO.: 15). * refers to the presence of a stabilized internucleotide linkage. _refers to the presence of a phosphodiester internucleotide linkage.

In yet other embodiments the oligonucleotide has only three YZ dinucleotides. The oligonucleotide may be T*C_G*T*T*T*T*G*A*C_G*T*T*T*T*G*T*C_G*T*T (SEQ ID NO.: 2), G*T*C_G*T*T*T*T*G*A*C_G*T*T*T*T*G*T*C_G*T*T (SEQ ID NO.: 8), T*C_G*T*C_G*T*T*T*T*G*A*C_G*T*T*T*T*G*T*C (SEQ ID NO.: 9), or T*C_G*T*C_G*T*T*T*T*G*A*C (SEQ ID NO.: 10). *refers to the presence of a stabilized internucleotide linkage. _refers to the presence of a phosphodiester internucleotide linkage.

According to other embodiments the oligonucleotide has only four YZ dinucleotides. The oligonucleotide may be T*C_G*T*C_G*T*T*T T*G*A*C_G*T*T*T*T*G*T*C_G*T*T (SEQ ID NO.: 4), T*C_G*T*C_G*T*T*T_T*G*A*C_G*T*T*T_T*G*T*C_G*T*T (SEQ ID NO.: 5), T*C_G*T*C_G*T_T*T_T*G_A*C_G*T_T*T_T*G_T*C_G*T*T (SEQ ID NO.: 6), C_G*T*C_G*T*T*T*T*G*A*C_G*T*T*T*T*G*T*C_G*T*T (SEQ ID NO.: 17), T*C_I*T*C_I*T*T*T*T*G*A*C_I*T*T*T*T*G*T*C_I*T*T (SEQ ID NO.: 18), T* MeC_G*T*MeC_G*T*T*T*T*G*A*MeC_G*T*T*T*T*G*T*MeC_G*T*T (SEQ ID NO.: 19), T*H_G*T*H_G*T*T*T*T*G*A*H_G*T*T*T*T*G*T*H_G*T*T (SEQ ID NO.: 20), T*C_{—}7*T*C_{—}7*T*T*T*T*G*A*C_{—}7*T*T*T*T*G*T*C_{—}7*T*T (SEQ ID NO.: 21), or U*C_G*U*C_G*U*U*U*U*G*A*C_G*U*U*U*U*G*U*C_G*U*U (SEQ ID NO.: 22). *refers to the presence of a stabilized internucleotide linkage. _refers to the presence of a phosphodiester internucleotide linkage. I is Inosine comprising a Hypoxanthine base; MeC is 5′-Methyl-Cytosine, H is 5-Hydroxy-Cytosine, 7 is 7-Deaza-Guanine, and U is Uracil.

Each YZ dinucleotide, in some embodiments has a phosphodiester or phosphodiester-like internucleotide linkage. The phosphodiester-like linkage, in some embodiments is boranophosphonate or diastereomerically pure Rp phosphorothioate.

The stabilized internucleotide linkages may be phosphorothioate, phosphorodithioate, methylphosphonate, methylphosphorothioate, ethylphosphate or any combination thereof.

In preferred embodiments Y is a nucleotide comprising an unmethylated cytosine and/or Z is a nucleotide comprising a guanine. Y optionally may be a nucleotide comprising a cytosine or a modified cytosine base such as 5-methyl cytosine, 5-methyl-isocytosine, 5-hydroxy-cytosine, 5-halogeno cytosine, uracil, N4-ethyl-cytosine, 5-fluoro-uracil, or hydrogen.

Optionally, Z may be a nucleotide comprising guanine or a modified guanine base such as 7-deazaguanine, 7-deaza-7-substituted guanine (such as 7-deaza-7-(C2-C6)alkynylguanine), 7-deaza-8-substituted guanine, hypoxanthine, 2,6-diaminopurine, 2-aminopurine, purine, 8-substituted guanine such as 8-hydroxyguanine, and 6-thioguanine, 2-aminopurine, or hydrogen.

In some embodiments the oligonucleotide has a 3′-3′ linkage with one or two accessible 5′ ends. In other embodiments the oligonucleotide has two accessible 5′ ends, each of which are 5′TCG.

An oligonucleotide of 2 to 7 nucleotides in length is provided according to other aspects of the invention. The oligonucleotide has at least one YZ dinucleotide with a phosphodiester or phosphodiester-like internucleotide linkage and the oligonucleotide includes at least one stabilized internucleotide linkage. Y is a nucleotide comprising a pyrimidine or modified pyrimidine base. Z is a nucleotide comprising a guanine or modified guanine.

In some embodiments the oligonucleotide has only one YZ dinucleotide. The oligonucleotide may be T*G*T*C*G*T*T (SEQ ID NO.: 23), T*G*T*C_G*T*T (SEQ ID NO.: 24), G*T*C*G*T*T (SEQ ID NO.: 25), G*T*C_G*T*T (SEQ ID NO.: 26), G*T*C*G*T (SEQ ID NO.: 27), G*T*C_G*T (SEQ ID NO.: 28), T*C*G*T*T (SEQ ID NO.: 29), T*C_G*T*T (SEQ ID NO.: 30), or C_G (SEQ ID NO.: 31). *refers to the presence of a stabilized internucleotide linkage. _refers to the presence of a phosphodiester internucleotide linkage. The stabilized internucleotide linkage may be phosphorothioate.

In some embodiments Y is a nucleotide comprising an unmethylated cytosine or a modified cytosine base selected from the group consisting of 5-methyl cytosine, 5-methyl-isocytosine, 5-hydroxy-cytosine, 5-halogeno cytosine, uracil, N4-ethyl-cytosine, 5-fluoro-uracil, or hydrogen. In other embodiments Z is guanine or a modified guanine base selected from the group consisting of 7-deazaguanine, 7-deaza-7-substituted guanine (such as 7-deaza-7-(C2-C6)alkynylguanine), 7-deaza-8-substituted guanine, hypoxanthine, 2,6-diaminopurine, 2-aminopurine, purine, 8-substituted guanine such as 8-hydroxyguanine, and 6-thioguanine, 2-aminopurine, or hydrogen.

In some embodiments the oligonucleotide has a 3′-3′ linkage with one or two accessible 5′ ends. In other embodiments the oligonucleotide has two accessible 5′ ends, each of which are 5′TCG.

In other embodiments the oligonucleotide has a 3′-aminohexyl group. In other embodiments the oligonucleotide has a 5′-aminohexyl group. In other embodiments the oligonucleotide has a 3′-aminohexyl group and a 5′-aminohexyl group.

In some embodiments the oligonucleotide has two YZ dinucleotides coupled by a spacer. In some embodiments the spacer consists of two hexaethyleneglycolgroups connected by a doubler. In some embodiments the doubler is a phosphoramidite. In some embodiments the amidite is a Symmetric Doubler Phosphoamidite (Glen Research Cat#10-1920-90). In some embodiments the amidite has a butyrate group attached to it. The oligonucleotide may be (C-G-L)-2doub-but (SEQ ID NO.: 43).

In other aspects the invention is an oligonucleotide of 7 nucleotides in length. The oligonucleotide has at least one CG dinucleotide and includes at least one stabilized internucleotide linkage. In some embodiments all of the internucleotide linkages are phosphorothioate linkages.

According to other aspects of the invention an oligonucleotide of 5 to 7 nucleotides in length is provided. The oligonucleotide has a GTCGT or TCGTT, and includes at least one stabilized internucleotide linkage. Optionally, all of the internucleotide linkages are phosphorothioate linkages.

Like fully stabilized immunostimulatory oligonucleotides, the immunostimulatory oligonucleotides of the instant invention are useful for inducing a Th1-like immune response. Accordingly, the immunostimulatory oligonucleotides of the instant invention are useful as adjuvants for vaccination, and they are useful for treating diseases including cancer, infectious disease, allergy, and asthma. They are believed to be of particular use in any condition calling for prolonged or repeated administration of immunostimulatory oligonucleotide for any purpose.

In another aspect, the invention is a method for treating allergy. The method is performed by administering to a subject having or at risk of having allergy an immunostimulatory CpG oligonucleotide described herein in an effective amount to treat allergy.

In another aspect, the invention is a method for treating asthma. The method is performed by administering to a subject having or at risk of having asthma an immunostimulatory CpG oligonucleotide described herein in an effective amount to treat asthma.

In one embodiment the oligonucleotide is administered to a mucosal surface. In other embodiments the oligonucleotide is administered in an aerosol formulation. Optionally the oligonucleotide is administered intranasally.

In another aspect the invention is a composition of the CpG immunostimulatory oligonucleotides described herein in combination with an antigen or other therapeutic compound, such as an anti-microbial agent. The anti-microbial agent may be, for instance, an anti-viral agent, an anti-parasitic agent, an anti-bacterial agent or an anti-fungal agent.

A composition of a sustained release device including the CpG immunostimulatory oligonucleotides described herein is provided according to another aspect of the invention.

The composition may optionally include a pharmaceutical carrier and/or be formulated in a delivery device. In some embodiments the delivery device is selected from the group consisting of cationic lipids, cell permeating proteins, and sustained release devices. In one embodiment the sustained release device is a biodegradable polymer or a microparticle.

According to another aspect of the invention a method of stimulating an immune response is provided. The method involves administering a CpG immunostimulatory oligonucleotide to a subject in an amount effective to induce an immune response in the subject. Preferably the CpG immunostimulatory oligonucleotide is administered orally, locally, in a sustained release device, mucosally, systemically, parenterally, or intramuscularly. When the CpG immunostimulatory oligonucleotide is administered to the mucosal surface it may be delivered in an amount effective for inducing a mucosal immune response or a systemic immune response. In preferred embodiments the mucosal surface is selected from the group consisting of an oral, nasal, rectal, vaginal, and ocular surface.

In some embodiments the method includes exposing the subject to an antigen wherein the immune response is an antigen-specific immune response. In some embodiments the antigen is selected from the group consisting of a tumor antigen, a viral antigen, a bacterial antigen, a parasitic antigen and a peptide antigen.

CpG immunostimulatory oligonucleotides are capable of provoking a broad spectrum of immune response. For instance these CpG immunostimulatory oligonucleotides can be used to redirect a Th2 to a Th1 immune response. CpG immunostimulatory oligonucleotides may also be used to activate an immune cell, such as a lymphocyte (e.g., B and T cells), a dendritic cell, and an NK cell. The activation can be performed in vivo, in vitro, or ex vivo, i.e., by isolating an immune cell from the subject, contacting the immune cell with an effective amount to activate the immune cell of the CpG immunostimulatory oligonucleotide and re-administering the activated immune cell to the subject. In some embodiments the dendritic cell presents a cancer antigen. The dendritic cell can be exposed to the cancer antigen ex vivo.

In still another embodiment, the CpG immunostimulatory oligonucleotides are useful for treating cancer. The CpG immunostimulatory oligonucleotides are also useful according to other aspects of the invention in preventing cancer (e.g., reducing a risk of developing cancer) in a subject at risk of developing a cancer. The cancer may be selected from the group consisting of biliary tract cancer, breast cancer, cervical cancer, choriocarcinoma, colon cancer, endometrial cancer, gastric cancer, intraepithelial neoplasms, lymphomas, liver cancer, lung cancer (e.g. small cell and non-small cell), melanoma, neuroblastomas, oral cancer, ovarian cancer, pancreatic cancer, prostate cancer, rectal cancer, sarcomas, thyroid cancer, and renal cancer, as well as other carcinomas and sarcomas. In some important embodiments, the cancer is selected from the group consisting of bone cancer, brain and CNS cancer, connective tissue cancer, esophageal cancer, eye cancer, Hodgkin's lymphoma, larynx cancer, oral cavity cancer, skin cancer, and testicular cancer.

CpG immunostimulatory oligonucleotides may also be used for increasing the responsiveness of a cancer cell to a cancer therapy (e.g., an anti-cancer therapy), optionally when the CpG immunostimulatory oligonucleotide is administered in conjunction with an anti-cancer therapy. The anti-cancer therapy may be a chemotherapy, a vaccine (e.g., an in vitro primed dendritic cell vaccine or a cancer antigen vaccine) or an antibody based therapy. This latter therapy may also involve administering an antibody specific for a cell surface antigen of, for example, a cancer cell, wherein the immune response results in antibody dependent cellular cytotoxicity (ADCC). In one embodiment, the antibody may be selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA.

Thus, according to some aspects of the invention, a subject having cancer or at risk of having a cancer is administered a CpG immunostimulatory oligonucleotide and an anti-cancer therapy. In some embodiments, the anti-cancer therapy is selected from the group consisting of a chemotherapeutic agent, an immunotherapeutic agent and a cancer vaccine.

In other aspects, the invention is a method for inducing an innate immune response by administering to the subject a CpG immunostimulatory oligonucleotide in an amount effective for activating an innate immune response.

According to another aspect of the invention a method for treating a viral or retroviral infection is provided. The method involves administering to a subject having or at risk of having a viral or retroviral infection, an effective amount for treating the viral or retroviral infection of any of the compositions of the invention. In some embodiments the virus is caused by a hepatitis virus e.g., hepatitis B, hepatitis C, HIV, herpes virus, or papillomavirus.

A method for treating bacterial infection is provided according to another aspect of the invention. The method involves administering to a subject having or at risk of having a bacterial infection, an effective amount for treating the bacterial infection of any of the compositions of the invention. In one embodiment the bacterial infection is due to an intracellular bacteria.

In another aspect the invention is a method for treating a parasite infection by administering to a subject having or at risk of having a parasite infection, an effective amount for treating the parasite infection of any of the compositions of the invention. In one embodiment the parasite infection is due to an intracellular parasite. In another embodiment the parasite infection is due to a non-helminthic parasite.

In some embodiments the subject is a human and in other embodiments the subject is a non-human vertebrate such as a dog, cat, horse, cow, pig, turkey, goat, fish, monkey, chicken, rat, mouse, or sheep.

In another aspect, the invention relates to a method for treating autoimmune disease by administering to a subject having or at risk of having an autoimmune disease an effective amount for treating or preventing the autoimmune disease of any of the compositions of the invention.

In other embodiments the oligonucleotide is delivered to the subject in an effective amount to induce cytokine expression. Optionally the cytokine is selected from the group consisting of IL-6, TNFα, IFNα, IFNγ and IP-10. In other embodiments the oligonucleotide is delivered to the subject in an effective amount to shift the immune response to a Th1 biased response from a Th2 biased response or to inhibit the development of a Th2 biased response.

The invention is some aspects is a method for treating airway remodeling, comprising: administering to a subject an oligonucleotide described herein, in an effective amount to treat airway remodeling in the subject. In one embodiment the subject has asthma, chronic obstructive pulmonary disease, or is a smoker. In other embodiments the subject is free of symptoms of asthma.

Use of an oligonucleotide of the invention for stimulating an immune response is also provided as an aspect of the invention.

A method for manufacturing a medicament of an oligonucleotide of the invention for stimulating an immune response is also provided.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 is a set of graphs depicting levels of interferon-alpha (pg/ml) secreted from human PBMC following exposure of these cells to the oligonucleotides listed by number along the top X-axis of the graph. The tested oligonucleotides shown in FIG. 1 include 1A=SEQ ID NO.: 1, 1B=SEQ ID NO.: 2, 1C=SEQ ID NO.: 3, 1D=SEQ ID NO.: 4, 1E=SEQ ID NO.: 5, 1F=SEQ ID NO.: 6, 1G=SEQ ID NO.: 7, 1H=SEQ ID NO.: 8, 1I=SEQ ID NO.: 9, 1J=SEQ ID NO.: 10, 1K=SEQ ID NO.: 11, 1L=SEQ ID NO.: 12, 1M=SEQ ID NO.: 13, 1N=SEQ ID NO.: 14, 1O=SEQ ID NO.: 15, 1P=SEQ ID NO.: 16, 1Q=SEQ ID NO.: 17, 1R=SEQ ID NO.: 18, 1S=SEQ ID NO.: 19, 1T=SEQ ID NO.: 20, 1U=SEQ ID NO.: 21, AND 1V=SEQ ID NO.: 22. The concentration of oligonucleotide used to produce a particular data point is depicted along the X-axis (μM). The data shown represents the mean of four to six donors. The absolute levels in pg/ml cannot be compared directly, as PBMC from different donors were used, which show variability among each other.

FIG. 2 is a set of graphs depicting a comparison of semi-soft and fully hardened short CpG ODN on IFN-alpha induction at different concentrations. SEQ ID NO.: 23 and 24 are shown in FIG. 2A. SEQ ID NO.: 25, 26, 27, 28, 29, 30, and 31 are shown in FIG. 2B.

FIG. 3 is a set of graphs showing the induction of TLR 9 at different ODN concentrations for five different ODNs: SEQ ID NO: 25 (circle), SEQ ID NO: 26 (inverted triangle), SEQ ID NO: 36 (square), SEQ ID NO: 37 (diamond), SEQ ID NO: 38 (triangle) and DOTAP only (hexagon). HEK293 cells stably expressing human TLR9 and an NFκB-luciferase reporter construct were incubated for 16 h with the indicated ODN concentrations. Cells were lysed and TLR9 activation was determined by assaying luciferase activity. Each data point was done in triplicate. FIG. 3B depicts experiments with ODNs precomplexed with DOTAP (N-[1-(2,3-Dioleoyloxy)propyl]-N,N,N-triethylammonium methylsulfate), while FIG. 3A shows the experiment without DOTAP.

FIG. 4 is a set of graphs depicting levels of cytokines secreted from human PBMC following exposure of these cells to ODNs with different stabilized internucleotide linkages: SEQ ID NO: 38 (circle), SEQ ID NO: 25 (inverted triangle), SEQ ID NO: 26 (square), SEQ ID NO: 36 (diamond) and SEQ ID NO: 37 (triangle). FIG. 4A shows the induction of IFN-γ secretion, while FIGS. 4B-4D depict the secretion of IL-10, IL-6 and IFN-γ respectively. The concentration of oligonucleotide used to produce a particular data point is depicted along the X-axis (μM). The data shown represents the mean of three donors.

FIG. 5 is a set of graphs depicting levels of cytokines secreted from human PBMC following exposure of these cells to ODN dinucleotides with different stabilized internucleotide linkages: SEQ ID NO: 38 (circle), SEQ ID NO: 40 (inverted triangle), SEQ ID NO: 41 (square), SEQ ID NO: 42 (diamond), SEQ ID NO: 39 (triangle) and SEQ ID NO: 31 (hexagon). FIG. 5A shows the induction of IFN-α secretion, while FIGS. 5B and 5C show the secretion of IL-10 and IL-6 respectively. The concentration of oligonucleotide used to produce a particular data point is depicted along the X-axis (μM). The data shown represents the mean of three donors.

FIG. 6 is a set of graphs depicting levels of cytokines secreted from human PBMC following exposure of these cells to ODN (C-G-L)-2doub-but (SEQ ID NO: 43; light circle), the positive control ODN (SEQ ID NO: 38; inverted triangle) or DOTAP only (dark circle). FIG. 6A shows the induction of IFN-α secretion, while FIGS. 6B and 6C show the secretion of IL-10 and IL-6 respectively. The concentration of oligonucleotide used to produce a particular data point is depicted along the X-axis (μM). The data shown represents the mean of three donors.

### DETAILED DESCRIPTION

Soft and semi-soft immunostimulatory oligonucleotides are provided according to the invention The immunostimulatory oligonucleotides of the invention described herein, in some embodiments have improved properties including similar or enhanced potency, reduced systemic exposure to the kidney, liver and spleen, and may have reduced reactogenicity at injection sites. Although applicant is not bound by a mechanism, it is believed that these improved properties are associated with the strategic placement within the immunostimulatory oligonucleotides of phosphodiester or phosphodiester-like “internucleotide linkages”. The term “internucleotide linkage” as used herein refers to the covalent backbone linkage joining two adjacent nucleotides in an oligonucleotide molecule. The covalent backbone linkage will typically be a modified or unmodified phosphate linkage, but other modifications are possible. Thus a linear oligonucleotide that is n nucleotides long has a total of n−1 internucleotide linkages. These covalent backbone linkages can be modified or unmodified in the immunostimulatory oligonucleotides according to the teachings of the invention.

Whereas it has previosly been recognized that fully stabilized immunostimulatory oligonucleotides less than 20 nucleotides long can have modest immunostimulatory activity compared with longer (e.g., 24 nucleotides long) fully stabilized oligonucleotides, semi-soft oligonucleotides as short as 16 nucleotides long have been discovered to have immunostimulatory activity at least equal to immunostimulatory activity of fully stabilized oligonucleotides over 20 nucleotides long. For example, SEQ ID NO: 32 and 33 (both 16-mers with partial sequence similarity to SEQ ID NO: 34) exhibit immunositmultory activity comparable to that of SEQ ID NO: 34 (24-mer). These ODN have the following sequences:
T*C_G*T*C_G*T*T*T*C_G*T*C_G*T*T (SEQ ID NO: 32),
T*C_G*T*C_G*T*T*T*T_G*T*C_G*T*T (SEQ ID NO: 33) and
T*C*G*T*C*G*T*T*T*T*G*T*C*G*T*T*T*T*G*T*C*G*T*T (SEQ ID NO: 34).

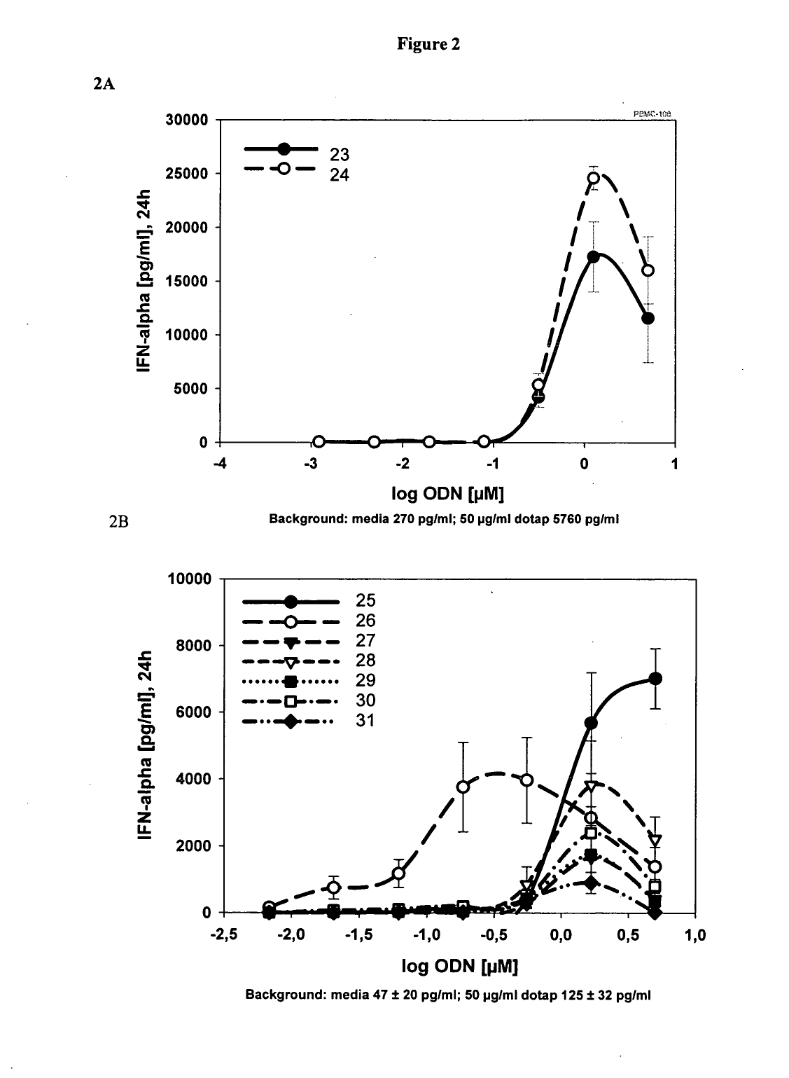

In some instances where a 5-mer phosphorothioate oligonucleotide appeared to lack immunostimulatory activity, substitution of even one phosphodiester internal YZ internucleotide linkage for a phosphorothioate linkage was found to yield a corresponding 5-mer with immunostimulatory activity (compare SEQ ID NO: 27 versus 28 in Table 3 and FIG. 2). At higher concentrations, in some instances even a 2-3-mer (i.e SEQ ID NO: 31) demonstrates activity. In other instances optimal oligonucleotides having either a soft, semi-soft or fully hardened backbone and having a length between 5 and 7 nucleotides have been identified. See for instance SEQ ID NO.: 27 and 29. The oligonucleotides with fully hardened backbone in this size range are particulary active at higher concentrations.

In particular, phosphodiester or phosphodiester-like internucleotide linkages involve “internal dinucleotides”. An internal dinucleotide in general shall mean any pair of adjacent nucleotides connected by an internucleotide linkage, in which neither nucleotide in the pair of nucleotides is a terminal nucleotide, i.e., neither nucleotide in the pair of nucleotides is a nucleotide defining the 5′ or 3′ end of the oligonucleotide. Thus a linear oligonucleotide that is n nucleotides long has a total of n−1 dinucleotides and only n−3 internal dinucleotides. Each internucleotide linkage in an internal dinucleotide is an internal internucleotide linkage. Thus a linear oligonucleotide that is n nucleotides long has a total of n−1 internucleotide linkages and only n−3 internal internucleotide linkages. The strategically placed phosphodiester or phosphodiester-like internucleotide linkages, therefore, refer to phosphodiester or phosphodiester-like internucleotide linkages positioned between any pair of nucleotides in the nucleic acid sequence. In some embodiments the phosphodiester or phosphodiester-like internucleotide linkages are not positioned between either pair of nucleotides closest to the 5′ or 3′ end.

The invention is based at least in some aspects on the surprising discovery that the soft and semi-soft oligonucleotides described herein have at least the same or in many cases possess greater immunostimulatory activity, in many instances, than corresponding fully stabilized immunostimulatory oligonucleotides having the same nucleotide sequence. It was further discovered that shorter oligonucleotides, e.g. 2-24 nucleotides in length retain immunostimulatory properties, even with the “softening” bond placed between the nucleotides of the CpG motif. This was unexpected because it is widely believed that phosphorothioate oligonucleotides are generally more immunostimulatory than unstabilized oligonucleotides. The results were surprising because it was expected that if the “softening” bond was placed between the critical immunostimulatory motif, i.e. CG that the oligonucleotide might have reduced activity because the oligonucleotide would easily be broken down into non-CG containing fragments in vivo.

A soft oligonucleotide is an immunostimulatory oligonucleotide having a partially stabilized backbone, in which phosphodiester or phosphodiester-like internucleotide linkages occur only within and immediately adjacent to at least one internal dinucleotide comprising pyrimidine -purine bases (YZ). Preferably YZ is YG, a dinucleotide comprising pyrimidine-guanine bases (YG). The at least one internal YZ dinucleotide itself has a phosphodiester or phosphodiester-like internucleotide linkage. A phosphodiester or phosphodiester-like internucleotide linkage occurring immediately adjacent to the at least one internal YZ dinucleotide can be 5′, 3′, or both 5′ and 3′ to the at least one internal YZ dinucleotide. Preferably a phosphodiester or phosphodiester-like internucleotide linkage occurring immediately adjacent to the at least one internal YZ dinucleotide is itself an internal internucleotide linkage. Thus for a sequence N₁YZ N₂, wherein N₁, and N₂ are each, independent of the other, any single nucleotide, the YZ dinucleotide has a phosphodiester or phosphodiester-like internucleotide linkage, and in addition (a) N₁, and Y are linked by a phosphodiester or phosphodiester-like internucleotide linkage when N₁ is an internal nucleotide, (b) Z and N₂ are linked by a phosphodiester or phosphodiester-like internucleotide linkage when N₂ is an internal nucleotide, or (c) N₁ and Y are linked by a phosphodiester or phosphodiester-like internucleotide linkage when N₁ is an internal nucleotide and Z and N₂ are linked by a phosphodiester or phosphodiester-like internucleotide linkage when N₂ is an internal nucleotide.

A semi-soft oligonucleotide is an immunostimulatory oligonucleotide having a partially stabilized backbone, in which phosphodiester or phosphodiester-like internucleotide linkages occur only within at least one internal dinucleotide comprising pyrimidine-purine bases (YZ). Semi-soft oligonucleotides generally possess increased immnunostimulatory potency relative to corresponding fully stabilized immunostimulatory oligonucleotides. For example, the immunostimulatory potency of semi-soft SEQ ID NO: 35 is 2-5 times that of all-phosphorothioate SEQ ID NO: 34, where the two oligonucleotides share the same nucleotide sequence and differ only as to internal YZ internucleotide linkages as follows, where * indicates phosphorothioate and _indicates phosphodiester:
T*C_G*T*C_G*T*T*T*T_G*T*C_G*T*T*T*T*G*T*C_G*T*T (SEQ ID NO:35) and
T*C*G*T*C*G*T*T*T*T*G*T*C*G*T*T*T*T*G*T*C*G*T*T (SEQ ID NO:34).
SEQ ID NO: 35 incorporates internal phophodiester internucleotide linkages involving both CG and TG (both YZ) dinucleotides. Due to the greater potency of semi-soft oligonucleotides, semi-soft oligonucleotides can be, in many instances, used at lower effective concentations and have lower effective doses than conventional fully stabilized immunostimulatory oligonucleotides in order to achieve a desired biological effect.

The oligonucleotides of the instant invention will generally include, in addition to the phosphodiester or phosphodiester-like internucleotide linkages at preferred internal positions, 5′ and 3′ ends that are resistant to degradation. Such degradation-resistant ends can involve any suitable modification that results in an increased resistance against exonuclease digestion over corresponding unmodified ends. For instance, the 5′ and 3′ ends can be stabilized by the inclusion there of at least one phosphate modification of the backbone. In a preferred embodiment, the at least one phosphate modification of the backbone at each end is independently a phosphorothioate, phosphorodithioate, methylphosphonate, ethylphosphate or methylphosphorothioate internucleotide linkage. In another embodiment, the degradation-resistant end includes one or more nucleotide units connected by peptide or amide linkages at the 3′ end. Yet other stabilized ends, including but not limited to those described further below, are meant to be encompassed by the invention.

As described above, the oligonucleotides of the instant invention include phosphodiester or phosphodiester-like linkages within and optionally adjacent to internal YZ dinucleotides. Such YZ dinucleotides are frequently part of immunostimulatory motifs. It is not necessary, however, that an oligonucleotide contain phosphodiester or phosphodiester-like linkages within every immunostimulatory motif.

A phosphodiester internucleotide linkage is the type of linkage characteristic of oligonucleotides found in nature. The phosphodiester internucleotide linkage includes a phosphorus atom flanked by two bridging oxygen atoms and bound also by two additional oxygen atoms, one charged and the other uncharged. Phosphodiester internucleotide linkage is particularly preferred when it is important to reduce the tissue half-life of the oligonucleotide.

A phosphodiester-like internucleotide linkage is a phosphorus-containing bridging group that is chemically and/or diastereomerically similar to phosphodiester. Measures of similarity to phosphodiester include susceptibility to nuclease digestion and ability to activate RNAse H. Thus for example phosphodiester, but not phosphorothioate, oligonucleotides are susceptible to nuclease digestion, while both phosphodiester and phosphorothioate oligonucleotides activate RNAse H. In a preferred embodiment the phosphodiester-like internucleotide linkage is boranophosphate (or equivalently, boranophosphonate) linkage. U.S. Pat. No. 5,177,198; U.S. Pat. No. 5,859,231; U.S. Pat. No. 6,160,109; U.S. Pat. No. 6,207,819; Sergueev et al., (1998) J Am Chem Soc 120:9417-27. In another preferred embodiment the phosphodiester-like internucleotide linkage is diasteromerically pure Rp phosphorothioate. It is believed that diasteromerically pure Rp phosphorothioate is more susceptible to nuclease digestion and is better at activating RNAse H than mixed or diastereomerically pure Sp phosphorothioate. It is to be noted that for purposes of the instant invention, the term “phosphodiester-like internucleotide linkage” specifically excludes phosphorodithioate and methylphosphonate internucleotide linkages.

The immunostimulatory oligonucleotide molecules of the instant invention may have a chimeric backbone. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. Since boranophosphonate linkages have been reported to be stabilized relative to phosphodiester linkages, for purposes of the chimeric nature of the backbone, boranophosphonate linkages can be classified either as phosphodiester-like or as stabilized, depending on the context. For example, a chimeric backbone according to the instant invention could in one embodiment include at least one phosphodiester (phosphodiester or phosphodiester-like) linkage and at least one boranophosphonate (stabilized) linkage. In another embodiment a chimeric backbone according to the instant invention could include boranophosphonate (phosphodiester or phosphodiester-like) and phosphorothioate (stabilized) linkages. A “stabilized internucleotide linkage” shall mean an internucleotide linkage that is relatively resistant to in vivo degradation (e.g., via an exo- or endo-nuclease), compared to a phosphodiester internucleotide linkage. Preferred stabilized internucleotide linkages include, without limitation, phosphorothioate, phosphorodithioate, methylphosphonate, ethylphosphate and methylphosphorothioate. Other stabilized internucleotide linkages include, without limitation: peptide, alkyl, dephospho, and others as described above.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described. Uhlmann E et al. (1990) Chem Rev 90:544; Goodchild J (1990) Bioconjugate Chem 1:165. Methods for preparing chimeric oligonucleotides are also known. For instance patents issued to Uhlmann et al have described such techniques.

Mixed backbone modified ODN may be synthesized using a commercially available DNA synthesizer and standard phosphoramidite chemistry. (F. E. Eckstein, “Oligonucleotides and Analogues—A Practical Approach” IRL Press Oxford, UK, 1991, and M. D. Matteucci and M. H. Caruthers, Tetrahedron Lett. 21, 719 (1980)) After coupling, PS linkages are introduced by sulfurization using the Beaucage reagent (R. P. Iyer, W. Egan, J. B. Regan and S. L. Beaucage, J. Am. Chem. Soc. 112, 1253 (1990)) (0.075 M in acetonitrile) or phenyl acetyl disulfide (PADS) followed by capping with acetic anhydride, 2,6-lutidine in tetrahydrofurane (1:1:8; v:v:v) and N-methylimidazole (16% in tetrahydrofurane). This capping step is performed after the sulfurization reaction to minimize formation of undesired phosphodiester (PO) linkages at positions where a phosphorothioate linkage should be located. In the case of the introduction of a phosphodiester linkage, e.g. at a CpG dinucleotide, the intermediate phosphorous-III is oxidized by treatment with a solution of iodine in water/pyridine. After cleavage from the solid support and final deprotection by treatment with concentrated ammonia (15 hrs at 50° C.), the ODN are analyzed by HPLC on a Gen-Pak Fax column (Millipore-Waters) using a NaCl-gradient (e.g. buffer A: 10 mM NaH₂PO₄ in acetonitrile/water=1:4/v:v pH 6.8; buffer B: 10 mM NaH₂PO₄, 1.5 M NaCl in acetonitrile/water=1:4/v:v; 5 to 60% B in 30 minutes at 1 ml/min) or by capillary gel electrophoresis. The ODN can be purified by HPLC or by FPLC on a Source High Performance column (Amersham Pharmacia). HPLC-homogeneous fractions are combined and desalted via a C18 column or by ultrafiltration. The ODN was analyzed by MALDI-TOF mass spectrometry to confirm the calculated mass.

The oligonucleotides of the invention can also include other modifications. These include nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Oligonucleotides which contain diol, such as tetraethyleneglycol or hexaethyleneglycol, at either or both termini have also been shown to be substantially resistant to nuclease degradation.

The oligonucleotides of the present invention are nucleic acids that contain specific sequences found to elicit an immune response. These specific sequences that elicit an immune response are referred to as “immunostimulatory motifs”, and the oligonucleotides that contain immunostimulatory motifs are referred to as “immunostimulatory nucleic acid molecules” and, equivalently, “immunostimulatory nucleic acids” or “immunostimulatory oligonucleotides”. The immunostimulatory oligonucleotides of the invention thus include at least one immunostimulatory motif. In a preferred embodiment the immunostimulatory motif is an “internal immunostimulatory motif”. The term “internal immunostimulatory motif” refers to the position of the motif sequence within a longer oligonucleotide sequence, which is longer in length than the motif sequence by at least one nucleotide linked to both the 5′ and 3′ ends of the immunostimulatory motif sequence.

In some embodiments of the invention the immunostimulatory oligonucleotides include immunostimulatory motifs which are “CpG dinucleotides”. A CpG dinucleotide can be methylated or unmethylated. An immunostimulatory oligonucleotide containing at least one unmethylated CpG dinucleotide is an oligonucleotide molecule which contains an unmethylated cytosine-guanine dinucleotide sequence (i.e., an unmethylated 5′ cytidine followed by 3′ guanosine and linked by a phosphate bond) and which activates the immune system; such an immunostimulatory oligonucleotide is a CpG oligonucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068. An immunostimulatory oligonucleotide containing at least one methylated CpG dinucleotide is an oligonucleotide which contains a methylated cytosine-guanine dinucleotide sequence (i.e., a methylated 5′ cytidine followed by a 3′ guanosine and linked by a phosphate bond) and which activates the immune system. In other embodiments the immunostimulatory oligonucleotides are free of CpG dinucleotides. These oligonucleotides which are free of CpG dinucleotides are referred to as non-CpG oligonucleotides, and they have non-CpG immunostimulatory motifs. The invention, therefore, also encompasses oligonucleotides with other types of immunostimulatory motifs, which can be methylated or unmethylated. The immunostimulatory oligonucleotides of the invention, further, can include any combination of methylated and unmethylated CpG and non-CpG immunostimulatory motifs.

As to CpG oligonucleotides, it has recently been described that there are different classes of CpG oligonucleotides. One class is potent for activating B cells but is relatively weak in inducing IFN-α and NK cell activation; this class has been termed the B class. The B class CpG nucleic acids typically are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068. Another class is potent for inducing IFN-α and NK cell activation but is relatively weak at stimulating B cells; this class has been termed the A class. The A class CpG nucleic acids typically have stabilized poly-G sequences at 5′ and 3′ ends and a palindromic phosphodiester CpG dinucleotide-containing sequence of at least 6 nucleotides. See, for example, published patent application PCT/US00/26527 (WO 01/22990). Yet another class of CpG nucleic acids activates B cells and NK cells and induces IFN-α; this class has been termed the C-class. The C-class CpG nucleic acids, as first characterized, typically are fully stabilized, include a B class-type sequence and a GC-rich palindrome or near-palindrome. This class has been described in co-pending U.S. patent application Ser. No. US10/224,523 filed on Aug. 19, 2002, the entire contents of which are incorporated herein by reference.

Thus, the invention in one aspect involves the finding that specific sub-classes of CpG immunostimulatory oligonucleotides having chimeric backbones are highly effective in mediating immune stimulatory effects. These CpG nucleic acids are useful therapeutically and prophylactically for stimulating the immune system to treat cancer, infectious diseases, allergy, asthma, autoimmune disease, and other disorders and to help protect against opportunistic infections following cancer chemotherapy. The strong yet balanced, cellular and humoral immune responses that result from CpG stimulation reflect the body's own natural defense system against invading pathogens and cancerous cells.

The invention involves, in one aspect, the discovery that a subset of CpG immunostimulatory oligonucleotides have improved immune stimulatory properties and reduced renal inflammatory effect. In some instances, renal inflammation has been observed in subjects that have been administered a completely phosphorothioate oligonucleotide. It is believed that the chimeric oligonucleotides described herein produce less renal inflammation than fully phosphorothioate oligonucleotides. Additionally these oligonucleotides are highly effective in stimulating an immune response. Thus, the phosphodiester region of the molecule did not reduce its affectivity.

The symbol * used in reference to an internucleotide bond of an oligonucleotide refers to the presence of a stabilized internucleotide linkage. The internucleotide linkages not marked with an * may be stabilized or unstabilized, as long as the oligonucleotide includes at least 2-3 phosphodiester internucleotide linkages. In some embodiments it is preferred that the oligonucleotides include 3-6 phosphodiester linkages. In some cases the linkages between the CG motifs are phosphodiester and in other cases they are phosphorothioate or other stabilized linkages.

The symbol _used in reference to an internucleotide bond of an oligonucleotide refers to the presence of a phosphodiester internucleotide linkage.

The terms “nucleic acid” and “oligonucleotide” also encompass nucleic acids or oligonucleotides with substitutions or modifications, such as in the bases and/or sugars. For example, they include oligonucleotides having backbone sugars that are covalently attached to low molecular weight organic groups other than a hydroxyl group at the 2′ position and other than a phosphate group or hydroxy group at the 5′ position. Thus modified oligonucleotides may include a 2′-O-alkylated ribose group. In addition, modified oligonucleotides may include sugars such as arabinose or 2′-fluoroarabinose instead of ribose. Thus the oligonucleotides may be heterogeneous in backbone composition thereby containing any possible combination of polymer units linked together such as peptide-nucleic acids (which have an amino acid backbone with nucleic acid bases).

Oligonucleotides also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, thymine, 5-methylcytosine, 5-hydroxycytosine, 5-fluorocytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art and many of which are described below.

The immunostimulatory oligonucleotides of the instant invention can encompass various chemical modifications and substitutions, in comparison to natural RNA and DNA, involving a phosphodiester internucleotide bridge, a β-D-ribose unit and/or a non-natural nucleotide base (adenine, guanine, cytosine, thymine, uracil). Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke ST et al. (1996) Annu Rev Pharmacol Toxicol 36:107-129; and Hunziker Jet al. (1995) Mod Synth Methods 7:331-417. An oligonucleotide according to the invention may have one or more modifications, wherein each modification is located at a particular phosphodiester internucleotide bridge and/or at a particular β-D-ribose unit and/or at a particular natural nucleotide base position in comparison to an oligonucleotide of the same sequence which is composed of natural DNA or RNA.

For example, the invention relates to an oligonucleotide which may comprise one or more modifications and wherein each modification is independently selected from:
a) the replacement of a phosphodiester internucleotide bridge located at the 3′ and/or the 5′ end of a nucleotide by a modified internucleotide bridge,
b) the replacement of phosphodiester bridge located at the 3′ and/or the 5′ end of a nucleotide by a dephospho bridge,
c) the replacement of a sugar phosphate unit from the sugar phosphate backbone by another unit,
d) the replacement of a β-D-ribose unit by a modified sugar unit, and
e) the replacement of a natural nucleotide base by a modified nucleotide base.

More detailed examples for the chemical modification of an oligonucleotide are as follows.

A phosphodiester internucleotide bridge located at the 3′ and/or the 5′ end of a nucleotide can be replaced by a modified internucleotide bridge, wherein the modified internucleotide bridge is for example selected from phosphorothioate, ethylphosphate phosphorodithioate, NR¹R²-phosphoramidate, boranophosphate, α-hydroxybenzyl phosphonate, phosphate-(C₁-C₂₁)-O-alkyl ester, phosphate-[(C₆-C₁₂)aryl-(C₁-C₂₁)-O-alkyl]ester, (C₁-C₈)alkylphosphonate and/or (C₆-C₁₂)arylphosphonate bridges, (C₇-C₁₂)-α-hydroxymethyl-aryl (e.g., disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleotide by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

A sugar phosphate unit (i.e., a β-D-ribose and phosphodiester internucleotide bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak EP et al. (1989) Nucleic Acids Res 17:6129-41), that is, e.g., the replacement by a morpholino-derivative unit; or to build up a polyamide nucleic acid (“PNA”; as described for example, in Nielsen PE et al. (1994) Bioconjug Chem 5:3-7), that is, e.g., the replacement by a PNA backbone unit, e.g., by 2-aminoethylglycine.

A β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O-(C₁-C₆)alkyl-ribose, preferably 2′-O-(C₁-C₆)alkyl-ribose is 2′-O-methylribose, 2′-O-(C₂-C₆)alkenyl-ribose, 2′-[O-(C₁-C₆)alkyl-O-(C₁-C₆)alkyl] -ribose, 2′-NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

In some embodiments the sugar is 2′-O-methylribose, particularly for one or both nucleotides linked by a phosphodiester or phosphodiester-like internucleotide linkage.

Oligonucleotides also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, and thymine, and other naturally and non-naturally occurring nucleobases, substituted and unsubstituted aromatic moieties.

A modified base is any base which is chemically distinct from the naturally occurring bases typically found in DNA and RNA such as T, C, G, A, and U, but which share basic chemical structures with these naturally occurring bases. The modified nucleotide base may be, for example, selected from hypoxanthine, uracil, dihydrouracil, pseudouracil, 2-thiouracil, 4-thiouracil, 5-aminouracil, 5-(C₁-C₆)-alkyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 2,4-diamino-purine, 8-azapurine, a substituted 7-deazapurine, preferably 7-deaza-7-substituted and/or 7-deaza-8-substituted purine, 5-hydroxymethylcytosine, N4-alkylcytosine, e.g., N4-ethylcytosine, 5-hydroxydeoxycytidine, 5-hydroxymethyldeoxycytidine, N4-alkyldeoxycytidine, e.g., N4-ethyldeoxycytidine, 6-thiodeoxyguanosine, and deoxyribonucleotides of nitropyrrole, C5-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleotide bases. This list is meant to be exemplary and is not to be interpreted to be limiting.

In particular formulas described herein a set of modified bases is defined. For instance the letter Y is used to refer to a nucleotide containing a cytosine or a modified cytosine. A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine. In another embodiment of the invention, the cytosine base is substituted by a universal base (e.g. 3-nitropyrrole, P-base), an aromatic ring system (e.g. fluorobenzene or difluorobenzene) or a hydrogen atom (dSpacer).

The letter Z is used to refer to guanine or a modified guanine base. A modified guanine as used herein is a naturally occurring or non-naturally occurring purine base analog of guanine which can replace this base without impairing the immunostimulatory activity of the oligonucleotide. Modified guanines include but are not limited to 7-deazaguanine, 7-deaza-7-substituted guanine (such as 7-deaza-7-(C2-C6)alkynylguanine), 7-deaza-8-substituted guanine, hypoxanthine, N2-substituted guanines (e.g. N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxo-adenine) 8-substituted guanine (e.g. 8-hydroxyguanine and 8-bromoguanine), and 6-thioguanine. In another embodiment of the invention, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom (dSpacer).

The oligonucleotides may have one or more accessible 5′ ends. It is possible to create modified oligonucleotides having two such 5′ ends. This may be achieved, for instance by attaching two oligonucleotides through a 3′-3′ linkage to generate an oligonucleotide having one or two accessible 5′ ends. The 3′3′-linkage may be a phosphodiester, phosphorothioate or any other modified internucleotide bridge. Methods for accomplishing such linkages are known in the art. For instance, such linkages have been described in Seliger, H.; et al., Oligonucleotide analogs with terminal 3′-3′- and 5′-5′-internucleotide linkages as antisense inhibitors of viral gene expression, Nucleotides & Nucleotides (1991), 10(1-3), 469-77 and Jiang, et al., Pseudo-cyclic oligonucleotides: in vitro and in vivo properties, Bioorganic & Medicinal Chemistry (1999), 7(12), 2727-2735.

The accessible 5′ and 3′ ends of the oligonucleotide may also be subsituted with an aminogroup. The aminogroup includes, but is not limited to, an aminohexyl residue.

Additionally, 3′3′-linked oligonucleotides where the linkage between the 3′-terminal nucleotides is not a phosphodiester, phosphorothioate or other modified bridge, can be prepared using an additional spacer, such as tri- or tetra-ethylenglycol phosphate moiety (Durand, M. et al, Triple-helix formation by an oligonucleotide containing one (dA)12 and two (dT)12 sequences bridged by two hexaethylene glycol chains, Biochemistry (1992), 31(38), 9197-204, U.S. Pat. No. 5,658,738, and U.S. Pat No. 5,668,265). Alternatively, the non-nucleotidic linker may be derived from ethanediol, propanediol, or from an abasic deoxyribose (dSpacer) unit (Fontanel, Marie Laurence et al., Sterical recognition by T4 polynucleotide kinase of non-nucleosidic moieties 5′-attached to oligonucleotides; Nucleic Acids Research (1994), 22(11), 2022-7) using standard phosphoramidite chemistry. The non-nucleotidic linkers can be incorporated once or multiple times, or combined with each other allowing for any desirable distance between the 3′-ends of the two ODNs to be linked.

For use in the instant invention, the oligonucleotides of the invention can be synthesized de novo using any of a number of procedures well known in the art. For example, the b-cyanoethyl phosphoramidite method (Beaucage, S. L., and Caruthers, M. H., Tet. Let. 22:1859, 1981); nucleotide H-phosphonate method (Garegg et al., Tet. Let. 27:4051-4054, 1986; Froehler et al., Nucl. Acid. Res. 14:5399-5407, 1986,; Garegg et al., Tet. Let. 27:4055-4058, 1986, Gaffney et al., Tet. Let. 29:2619-2622, 1988). These chemistries can be performed by a variety of automated nucleic acid synthesizers available in the market. These oligonucleotides are referred to as synthetic oligonucleotides. An isolated oligonucleotide generally refers to an oligonucleotide which is separated from components which it is normally associated with in nature. As an example, an isolated oligonucleotide may be one which is separated from a cell, from a nucleus, from mitochondria or from chromatin.

It has been discovered according to the invention that the subsets of CpG immunostimulatory oligonucleotides described herein have dramatic immune stimulatory effects on human cells such as NK cells, suggesting that these CpG immunostimulatory oligonucleotides are effective therapeutic agents for human vaccination, cancer immunotherapy, asthma immunotherapy, general enhancement of immune function, enhancement of hematopoietic recovery following radiation or chemotherapy, autoimmune disease and other immune modulatory applications.

Thus the CpG immunostimulatory oligonucleotides are useful in some aspects of the invention as a vaccine for the treatment of a subject at risk of developing allergy or asthma, an infection with an infectious organism or a cancer in which a specific cancer antigen has been identified. The CpG immunostimulatory oligonucleotides can also be given without the antigen or allergen for protection against infection, allergy or cancer, and in this case repeated doses may allow longer term protection. A subject at risk as used herein is a subject who has any risk of exposure to an infection causing pathogen or a cancer or an allergen or a risk of developing cancer. For instance, a subject at risk may be a subject who is planning to travel to an area where a particular type of infectious agent is found or it may be a subject who through lifestyle or medical procedures is exposed to bodily fluids which may contain infectious organisms or directly to the organism or even any subject living in an area where an infectious organism or an allergen has been identified. Subjects at risk of developing infection also include general populations to which a medical agency recommends vaccination with a particular infectious organism antigen. If the antigen is an allergen and the subject develops allergic responses to that particular antigen and the subject may be exposed to the antigen, i.e., during pollen season, then that subject is at risk of exposure to the antigen. A subject at risk of developing an allergy or asthma includes those subjects that have been identified as having an allergy or asthma but that don't have the active disease during the CpG immunostimulatory oligonucleotide treatment as well as subjects that are considered to be at risk of developing these diseases because of genetic or environmental factors.

A subject at risk of developing a cancer is one who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission. When a subject at risk of developing a cancer is treated with an antigen specific for the type of cancer to which the subject is at risk of developing and a CpG immunostimulatory oligonucleotide, the subject may be able to kill the cancer cells as they develop. If a tumor begins to form in the subject, the subject will develop a specific immune response against the tumor antigen.

In addition to the use of the CpG immunostimulatory oligonucleotides for prophylactic treatment, the invention also encompasses the use of the CpG immunostimulatory oligonucleotides for the treatment of a subject having an infection, an allergy, asthma, or a cancer.

A subject having an infection is a subject that has been exposed to an infectious pathogen and has acute or chronic detectable levels of the pathogen in the body. The CpG immunostimulatory oligonucleotides can be used with or without an antigen to mount an antigen specific systemic or mucosal immune response that is capable of reducing the level of or eradicating the infectious pathogen. An infectious disease, as used herein, is a disease arising from the presence of a foreign microorganism in the body. It is particularly important to develop effective vaccine strategies and treatments to protect the body's mucosal surfaces, which are the primary site of pathogenic entry.

A subject having an allergy is a subject that has or is at risk of developing an allergic reaction in response to an allergen. An allergy refers to acquired hypersensitivity to a substance (allergen). Allergic conditions include but are not limited to eczema, allergic rhinitis or coryza, hay fever, conjunctivitis, bronchial asthma, urticaria (hives) and food allergies, and other atopic conditions.

Allergies are generally caused by IgE antibody generation against harmless allergens. The cytokines that are induced by systemic or mucosal administration of CpG immunostimulatory oligonucleotides are predominantly of a class called Th1 (examples are IL-12, IP-10, IFN-α and IFN-γ) and these induce both humoral and cellular immune responses. The other major type of immune response, which is associated with the production of IL-4 and IL-5 cytokines, is termed a Th2 immune response. In general, it appears that allergic diseases are mediated by Th2 type immune responses. Based on the ability of the CpG immunostimulatory oligonucleotides to shift the immune response in a subject from a predominant Th2 (which is associated with production of IgE antibodies and allergy) to a balanced Th2/Th1 response (which is protective against allergic reactions), an effective dose for inducing an immune response of a CpG immunostimulatory oligonucleotide can be administered to a subject to treat or prevent asthma and allergy.

Thus, the CpG immunostimulatory oligonucleotides have significant therapeutic utility in the treatment of allergic and non-allergic conditions such as asthma. Th2 cytokines, especially IL-4 and IL-5 are elevated in the airways of asthmatic subjects. These cytokines promote important aspects of the asthmatic inflammatory response, including IgE isotope switching, eosinophil chemotaxis and activation and mast cell growth. Th1 cytokines, especially IFN-γ and IL-12, can suppress the formation of Th2 clones and production of Th2 cytokines. Asthma refers to a disorder of the respiratory system characterized by inflammation, narrowing of the airways and increased reactivity of the airways to inhaled agents. Asthma is frequently, although not exclusively associated with atopic or allergic symptoms.

A subject having a cancer is a subject that has detectable cancerous cells. The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma.

A subject shall mean a human or vertebrate animal including but not limited to a dog, cat, horse, cow, pig, sheep, goat, turkey, chicken, primate, e.g., monkey, and fish (aquaculture species), e.g. salmon. Thus, the invention can also be used to treat cancer and tumors, infections, and allergy/asthma in non human subjects. Cancer is one of the leading causes of death in companion animals (i.e., cats and dogs).

As used herein, the term treat, treated, or treating when used with respect to an disorder such as an infectious disease, cancer, allergy, or asthma refers to a prophylactic treatment which increases the resistance of a subject to development of the disease (e.g., to infection with a pathogen) or, in other words, decreases the likelihood that the subject will develop the disease (e.g., become infected with the pathogen) as well as a treatment after the subject has developed the disease in order to fight the disease (e.g., reduce or eliminate the infection) or prevent the disease from becoming worse.

In the instances when the CpG oligonucleotide is administered with an antigen, the subject may be exposed to the antigen. As used herein, the term exposed to refers to either the active step of contacting the subject with an antigen or the passive exposure of the subject to the antigen in vivo. Methods for the active exposure of a subject to an antigen are well-known in the art. In general, an antigen is administered directly to the subject by any means such as intravenous, intramuscular, oral, transdermal, mucosal, intranasal, intratracheal, or subcutaneous administration. The antigen can be administered systemically or locally. Methods for administering the antigen and the CpG immunostimulatory oligonucleotide are described in more detail below. A subject is passively exposed to an antigen if an antigen becomes available for exposure to the immune cells in the body. A subject may be passively exposed to an antigen, for instance, by entry of a foreign pathogen into the body or by the development of a tumor cell expressing a foreign antigen on its surface.

The methods in which a subject is passively exposed to an antigen can be particularly dependent on timing of administration of the CpG immunostimulatory oligonucleotide. For instance, in a subject at risk of developing a cancer or an infectious disease or an allergic or asthmatic response, the subject may be administered the CpG immunostimulatory oligonucleotide on a regular basis when that risk is greatest, i.e., during allergy season or after exposure to a cancer causing agent. Additionally the CpG immunostimulatory oligonucleotide may be administered to travelers before they travel to foreign lands where they are at risk of exposure to infectious agents. Likewise the CpG immunostimulatory oligonucleotide may be administered to soldiers or civilians at risk of exposure to biowarfare to induce a systemic or mucosal immune response to the antigen when and if the subject is exposed to it.

An antigen as used herein is a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and muticellular organisms such as parasites and allergens. The term antigen broadly includes any type of molecule which is recognized by a host immune system as being foreign. Antigens include but are not limited to cancer antigens, microbial antigens, and allergens.

A cancer antigen as used herein is a compound, such as a peptide or protein, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

A microbial antigen as used herein is an antigen of a microorganism and includes but is not limited to virus, bacteria, parasites, and fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of fungi include Cryptococcus neoformans, Histoplasma capsulatum, Coccidioides immitis, Blastomyces dermatitidis, Chlamydia trachomatis, Candida albicans.

Other infectious organisms (i.e., protists) include Plasmodium spp. such as Plasmodiumfalciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas'disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

An allergen refers to a substance (antigen) that can induce an allergic or asthmatic response in a susceptible subject. The list of allergens is enormous and can include pollens, insect venoms, animal dander dust, fimgal spores and drugs (e.g. penicillin). Examples of natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Loliumperenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinaceal); Paspalum (e.g. Paspalum notatum); Sorghum (e.g. Sorghum halepensis); and Bromus (e.g. Bromus inermis).

The term substantially purified as used herein refers to a polypeptide which is substantially free of other proteins, lipids, carbohydrates or other materials with which it is naturally associated. One skilled in the art can purify viral or bacterial polypeptides using standard techniques for protein purification. The substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the viral or bacterial polypeptide can also be determined by amino-terminal amino acid sequence analysis. Other types of antigens not encoded by a nucleic acid vector such as polysaccharides, small molecule, mimics etc are included within the invention.

The oligonucleotides of the invention may be administered to a subject with an anti-microbial agent. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fingal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial fimctions or structures which are specific for the microorganism and which are not present in host cells. Anti-viral agents can be isolated from natural sources or synthesized and are useful for killing or inhibiting viruses. Anti-fungal agents are used to treat superficial ftungal infections as well as opportunistic and primary systemic ftungal infections. Anti-parasite agents kill or inhibit parasites.

Examples of anti-parasitic agents, also referred to as parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Antibacterial agents kill or inhibit the growth or function of bacteria. A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics. Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors.

Antiviral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, zidovudine (azidothymidine), imiquimod, and resimiquimod.

The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Anti-viral agents useful in the invention include but are not limited to immunoglobulins, amantadine, interferons, nucleotide analogues, and protease inhibitors. Specific examples of anti-virals include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

CpG immunostimulatory oligonucleotides can be combined with other therapeutic agents such as adjuvants to enhance immune responses. The CpG immunostimulatory oligonucleotide and other therapeutic agent may be administered simultaneously or sequentially. When the other therapeutic agents are administered simultaneously they can be administered in the same or separate formulations, but are administered at the same time. The other therapeutic agents are administered sequentially with one another and with CpG immunostimulatory oligonucleotide, when the administration of the other therapeutic agents and the CpG immunostimulatory oligonucleotide is temporally separated. The separation in time between the administration of these compounds may be a matter of minutes or it may be longer. Other therapeutic agents include but are not limited to adjuvants, cytokines, antibodies, antigens, etc.

The compositions of the invention may also be administered with non-nucleic acid adjuvants. A non-nucleic acid adjuvant is any molecule or compound except for the CpG immunostimulatory oligonucleotides described herein which can stimulate the humoral and/or cellular immune response. Non-nucleic acid adjuvants include, for instance, adjuvants that create a depo effect, immune stimulating adjuvants, and adjuvants that create a depo effect and stimulate the immune system.

The CpG immunostimulatory oligonucleotides are also useful as mucosal adjuvants. It has previously been discovered that both systemic and mucosal immunity are induced by mucosal delivery of CpG oligonucleotides. Thus, the oligonucleotides may be administered in combination with other mucosal adjuvants.

Immune responses can also be induced or augmented by the co-administration or co-linear expression of cytokines (Bueler & Mulligan, 1996; Chow et al., 1997; Geissler et al., 1997; Iwasaki et al., 1997; Kim et al., 1997) or B-7 co-stimulatory molecules (Iwasaki et al., 1997; Tsuji et al, 1997) with the CpG immunostimulatory oligonucleotides. The term cytokine is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IL-1, IL-2, IL-4, IL-5, IL-6, IL-7, IL-10, IL-12, IL-1 5, IL-18, granulocyte-macrophage colony stimulating factor (GM-CSF), granulocyte colony stimulating factor (G-CSF), interferon-γ (γ-IFN), IFN-α, tumor necrosis factor (TNF), TGF-β, FLT-3 ligand, and CD40 ligand.

The oligonucleotides are also useful for redirecting an immune response from a Th2 immune response to a Th1 immune response. This results in the production of a relatively balanced Th1/Th2 environment. Redirection of an immune response from a Th2 to a Th1 immune response can be assessed by measuring the levels of cytokines produced in response to the oligonucleotide (e.g., by inducing monocytic cells and other cells to produce Th1 cytokines, including IL-12, IFN-γ and GM-CSF). The redirection or rebalance of the immune response from a Th2 to a Th1 response is particularly useful for the treatment or prevention of asthma. For instance, an effective amount for treating asthma can be that amount; useful for redirecting a Th2 type of immune response that is associated with asthma to a Th1 type of response or a balanced Th1/Th2 environment. Th2 cytokines, especially IL-4 and IL-5 are elevated in the airways of asthmatic subjects. The CpG immunostimulatory oligonucleotides of the invention cause an increase in Th1 cytokines which helps to rebalance the immune system, preventing or reducing the adverse effects associated with a predominately Th2 immune response.

The oligonucleotides are also useful for improving survival, differentiation, activation and maturation of dendritic cells. The CpG immunostimulatory oligonucleotides have the unique capability to promote cell survival, differentiation, activation and maturation of dendritic cells.

CpG immunostimulatory oligonucleotides also increase natural killer cell lytic activity and antibody dependent cellular cytotoxicity (ADCC). ADCC can be performed using a CpG immunostimulatory oligonucleotide in combination with an antibody specific for a cellular target, such as a cancer cell. When the CpG immunostimulatory oligonucleotide is administered to a subject in conjunction with the antibody the subject's immune system is induced to kill the tumor cell. The antibodies useful in the ADCC procedure include antibodies which interact with a cell in the body. Many such antibodies specific for cellular targets have been described in the art and many are commercially available.

The CpG immunostimulatory oligonucleotides may also be administered in conjunction with an anti-cancer therapy. Anti-cancer therapies include cancer medicaments, radiation and surgical procedures. As used herein, a “cancer medicament” refers to a agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. For the purpose of this specification, cancer medicaments are classified as chemotherapeutic agents, immunotherapeutic agents, cancer vaccines, hormone therapy, and biological response modifiers.

Additionally, the methods of the invention are intended to embrace the use of more than one cancer medicament along with the CpG immunostimulatory oligonucleotides. As an example, where appropriate, the CpG immunostimulatory oligonucleotides may be administered with both a chemotherapeutic agent and an immunotherapeutic agent. Alternatively, the cancer medicament may embrace an immunotherapeutic agent and a cancer vaccine, or a chemotherapeutic agent and a cancer vaccine, or a chemotherapeutic agent, an immunotherapeutic agent and a cancer vaccine all administered to one subject for the purpose of treating a subject having a cancer or at risk of developing a cancer.

The chemotherapeutic agent may be selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS famesyl transferase inhibitor, famesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZDO1O1, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, lodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabino side, Taxane Analog, nitrosoureas, alkylating agents such as melphelan and cyclophosphamide, Aminoglutethimide, Asparaginase, Busulfan, Carboplatin, Chlorombucil, Cytarabine HCI, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate, but it is not so limited.

The immunotherapeutic agent may be selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10Ab, SMART ABL 364 Ab and ImmuRAIT-CEA, but it is not so limited.

The cancer vaccine may be selected from the group consisting of EGF, Anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vacine, TA-HPV, TA-CIN, DISC-virus and ImmuCyst/TheraCys, but it is not so limited.

The use of CpG immunostimulatory oligonucleotides in conjunction with immunotherapeutic agents such as monoclonal antibodies is able to increase long-term survival through a number of mechanisms including significant enhancement of ADCC (as discussed above), activation of natural killer (NK) cells and an increase in IFNα levels. The oligonucleotides when used in combination with monoclonal antibodies serve to reduce the dose of the antibody required to achieve a biological result.

As used herein, the terms “cancer antigen” and “tumor antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses.

The CpG immunostimulatory oligonucleotides are also useful for treating and preventing autoimmune disease. Autoimmune disease is a class of diseases in which a subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

A “self-antigen” as used herein refers to an antigen of a normal host tissue. Normal host tissue does not include cancer cells. Thus an immune response mounted against a self-antigen, in the context of an autoimmune disease, is an undesirable immune response and contributes to destruction and damage of normal tissue, whereas an immune response mounted against a cancer antigen is a desirable immune response and contributes to the destruction of the tumor or cancer. Thus, in some aspects of the invention aimed at treating autoimmune disorders it is not recommended that the CpG immunostimulatory oligonucleotides be administered with self antigens, particularly those that are the targets of the autoimmune disorder.

In other instances, the CpG immunostimulatory oligonucleotides may be delivered with low doses of self-antigens. A number of animal studies have demonstrated that mucosal administration of low doses of antigen can result in a state of immune hyporesponsiveness or “tolerance.” The active mechanism appears to be a cytokine-mediated immune deviation away from a Th1 towards a predominantly Th2 and Th3 (i.e., TGF-β dominated) response. The active suppression with low dose antigen delivery can also suppress an unrelated immune response (bystander suppression) which is of considerable interest in the therapy of autoimmune diseases, for example, rheumatoid arthritis and SLE. Bystander suppression involves the secretion of Th1-counter-regulatory, suppressor cytokines in the local environment where proinflammatory and Th1 cytokines are released in either an antigen-specific or antigen-nonspecific manner. “Tolerance” as used herein is used to refer to this phenomenon. Indeed, oral tolerance has been effective in the treatment of a number of autoimmune diseases in animals including: experimental autoimmune encephalomyelitis (EAE), experimental autoimmune myasthenia gravis, collagen-induced arthritis (CIA), and insulin-dependent diabetes mellitus. In these models, the prevention and suppression of autoimmune disease is associated with a shift in antigen-specific humoral and cellular responses from a Th1 to Th2/Th3 response.

The invention also includes a method for inducing antigen non-specific innate immune activation and broad spectrum resistance to infectious challenge using the CpG immunostimulatory oligonucleotides. The term antigen non-specific innate immune activation as used herein refers to the activation of immune cells other than B cells and for instance can include the activation of NK cells, T cells or other immune cells that can respond in an antigen independent fashion or some combination of these cells. A broad spectrum resistance to infectious challenge is induced because the immune cells are in active form and are primed to respond to any invading compound or microorganism. The cells do not have to be specifically primed against a particular antigen. This is particularly useful in biowarfare, and the other circumstances described above such as travelers.

The CpG immunostimulatory oligonucleotides may be directly administered to the subject or may be administered in conjunction with a nucleic acid delivery complex. A nucleic acid delivery complex shall mean a nucleic acid molecule associated with (e.g. ionically or covalently bound to; or encapsulated within) a targeting means (e.g. a molecule that results in higher affinity binding to target cell. Examples of nucleic acid delivery complexes include nucleic acids associated with a sterol (e.g. cholesterol), a lipid (e.g. a cationic lipid, virosome or liposome), or a target cell specific binding agent (e.g. a ligand recognized by target cell specific receptor). Preferred complexes may be sufficiently stable in vivo to prevent significant uncoupling prior to internalization by the target cell. However, the complex can be cleavable under appropriate conditions within the cell so that the oligonucleotide is released in a functional form.

The term effective amount of a CpG immunostimulatory oligonucleotide refers to the amount necessary or sufficient to realize a desired biologic effect. For example, an effective amount of a CpG immunostimulatory oligonucleotide administered with an antigen for inducing mucosal immunity is that amount necessary to cause the development of IgA in response to an antigen upon exposure to the antigen, whereas that amount required for inducing systemic immunity is that amount necessary to cause the development of IgG in response to an antigen upon exposure to the antigen. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular CpG immunostimulatory oligonucleotide being administered the size of the subject, or the severity of the disease or condition. One of ordinary skill in the art can empirically determine the effective amount of a particular CpG immunostimulatory oligonucleotide and/or antigen and/or other therapeutic agent without necessitating undue experimentation.

Subject doses of the compounds described herein for mucosal or local delivery typically range from about 0.1 μg to 50 mg per administration, which depending on the application could be given daily, weekly, or monthly and any other amount of time therebetween. More typically mucosal or local doses range from about 10 μg to 10 mg per administration, and optionally from about 100 μg to 1 mg, with 2-4 administrations being spaced days or weeks apart. More typically, immune stimulant doses range from 1 μg to 10 mg per administration, and most typically 10 μg to 1 mg, with daily or weekly administrations. Subject doses of the compounds described herein for parenteral delivery for the purpose of inducing an antigen-specific immune response, wherein the compounds are delivered with an antigen but not another therapeutic agent are typically 5 to 10,000 times higher than the effective mucosal dose for vaccine adjuvant or immune stimulant applications, and more typically 10 to 1,000 times higher, and most typically 20 to 100 times higher. Doses of the compounds described herein for parenteral delivery e.g., for inducing an innate immune response, for increasing ADCC, for inducing an antigen specific immune response when the CpG immunostimulatory oligonucleotides are administered in combination with other therapeutic agents or in specialized delivery vehicles typically range from about 0.1 μg to 10 mg per administration, which depending on the application could be given daily, weekly, or monthly and any other amount of time therebetween. More typically parenteral doses for these purposes range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

For any compound described herein the therapeutically effective amount can be initially determined from animal models. A therapeutically effective dose can also be determined from human data for CpG oligonucleotides which have been tested in humans (human clinical trials have been initiated) and for compounds which are known to exhibit similar pharmacological activities, such as other adjuvants, e.g., LT and other antigens for vaccination purposes. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the CpG immunostimulatory oligonucleotide can be administered to a subject by any mode that delivers the oligonucleotide to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, and rectal.

For oral administration, the compounds (i.e., CpG immunostimulatory oligonucleotides, antigens and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the oligonucleotide (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fme multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the oligonucleotide (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the oligonucleotide or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the oligonucleotides (or derivatives thereof). The oligonucleotide (or derivative) is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin−1); Hubbard et al., 1989, Annals of Internal Medicine, Vol. III, pp. 206-212 (al- antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of oligonucleotide (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified oligonucleotide may also be prepared in different formulations depending on the type of chemical modification or the type of device employed.

Formulations suitable for use with a nebulizer, either jet or ultrasonic, will typically comprise oligonucleotide (or derivative) dissolved in water at a concentration of about 0.1 to 25 mg of biologically active oligonucleotide per mL of solution. The formulation may also include a buffer and a simple sugar (e.g., for oligonucleotide stabilization and regulation of osmotic pressure). The nebulizer formulation may also contain a surfactant, to reduce or prevent surface induced aggregation of the oligonucleotide caused by atomization of the solution in forming the aerosol.

Formulations for use with a metered-dose inhaler device will generally comprise a finely divided powder containing the oligonucleotide (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing oligonucleotide (or derivative) and may also include a bulking agent, such as lactose, sorbitol, sucrose, or mannitol in amounts which facilitate dispersal of the powder from the device, e.g., 50 to 90% by weight of the formulation. The oligonucleotide (or derivative) should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infuision. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The CpG immunostimulatory oligonucleotides and optionally other therapeutics and/or antigens may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a CpG immunostimulatory oligonucleotide and optionally antigens and/or other therapeutic agents optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

It recently has been reported that CpG oligonucleotides appear to exert their immunostimulatory effect through interaction with Toll-like receptor 9 (TLR9). Hemmi H et al. (2000) Nature 408:740-5. TLR9 signaling activity thus can be measured in response to CpG oligonucleotide or other immunostimulatory oligonucleotide by measuring NF-κB, NF-κB-related signals, and suitable events and intermediates upstream of NF-κB.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

Materials and Methods:

Oligodeoxynucleotides (ODNs) and Reagents

All ODN were purchased from Biospring (Frankfurt, Germany) or provided by Coley Pharmaceutical GmbH (Langenfeld, Germany), controlled for identity and purity by Coley Pharmaceutical GmbH and had undetectable endotoxin levels (<0.1 EU/ml) measured by the Limulus assay (BioWhittaker, Verviers, Belgium). ODN were suspended in sterile, endotoxin-free Tris-EDTA (Sigma, Deisenhofen, Germany), and stored and handled under aseptic conditions to prevent both microbial and endotoxin contamination. All dilutions were carried out using endotoxin-free Tris-EDTA.

TLR assays HEK293 cells were transfected by electroporation with vectors expressing the human TLR9 and a 6xNF-κB-luciferase reporter plasmid. Stable transfectants (3×10⁴ cells/well) were incubated with ODN for 16 h at 37° C. in a humidified incubator. Each data point was done in triplicate. Cells were lysed and assayed for luciferase gene activity (using the BriteLite kit from Perkin-Elmer, Zaventem, Belgium). Stimulation indices were calculated in reference to reporter gene activity of medium without addition of ODN.

Cell Purification

Peripheral blood buffy coat preparations from healthy human donors were obtained from the Blood Bank of the University of Düisseldorf (Germany) and PBMC were purified by centrifugation over Ficoll-Hypaque (Sigma). Cells were cultured in a humidified incubator at 37° C. in RPMI 1640 medium supplemented with 5% (v/v) heat inactivated human AB serum (BioWhittaker) or 10% (v/v) heat inactivated FCS, 2 mM L-glutamine, 100 U/ml penicillin and 100 μg/ml streptomycin (all from Sigma).

Cytokine detection and Flow Cytometric Analysis

PBMC were resuspended and added to 96 well round-bottomed plates. PBMC were incubated with various ODN concentrations and culture supernatants (SN) were collected after the indicated time points. If not used immediately, SN were stored at −20° C. until required.

Amounts of cytokines in the SN were assessed using commercially available ELISA kits for IFN-γ, IL-6 and IL-10 (Diaclone, Besangon, France), or an in-house ELISA for IFN-α, developed using commercially available antibodies (PBL, New Brunswick, N.J., USA).

### Example 1

### Ability of Short Semi-soft CpG ODN to Induce IFN-α Expression from Human PBMC

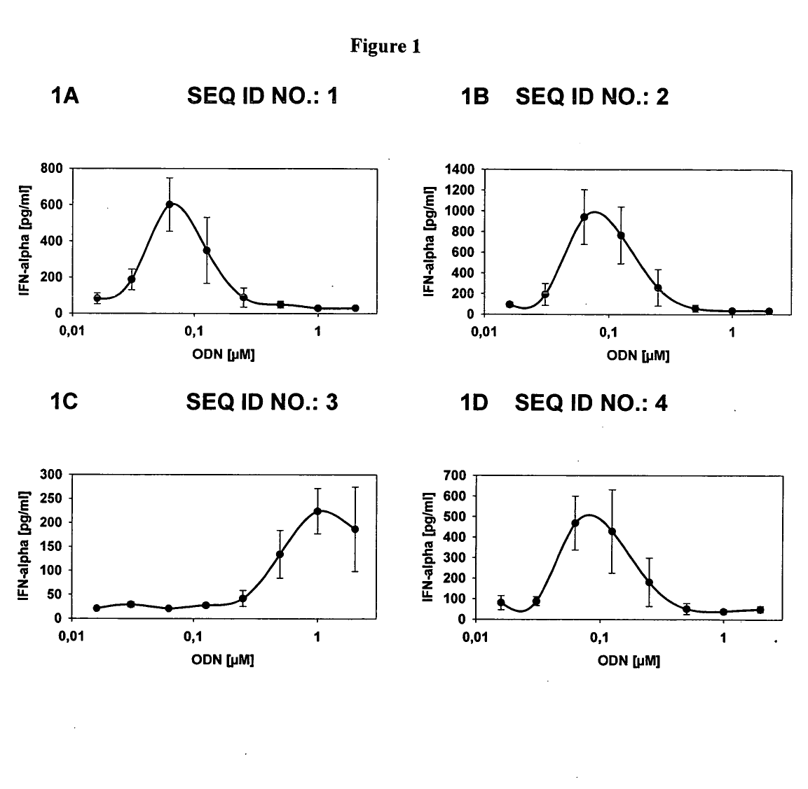

Levels of interferon-alpha (IFN-α) secreted from human PBMC following exposure of these cells to the CpG oligonucleotides described herein is shown in the attached FIG. 1. The test oligonucleotides examined are depicted in the figures by SEQ ID NO. The concentration of oligonucleotide used to produce a particular data point is depicted along the X-axis (μM).

As demonstrated in FIG. 1 each of the oligonucleotides examined in the assays were able to produce significant IFN-α secretion. A fully phosphodiester ODN (SEQ ID NO. 7) caused the production of only background levels of IFN-α.

A table describing the ODN used in the study is presented below (Table 1).

TABLE 1
ODN list

| | | | |
|---|---|---|---|
| SEQ ID | ODN Sequence | length | comments |

| | | | |
|---|---|---|---|
| 1 & | T*C_G*T*C_G*T*T*T*T*G*A*C_G*T*T*T*T*G*T*C_G*T*T | 24 | |
| 2 | T*C_G*T*T*T*T*G*A*C_G*T*T*T*T*G*T*C_G*T*T | 21 | 5′N-3 |
| 3 | T*C_G*T*T*T*T*G*A*C_G*T*T | 13 | 5′N-3, 3′N-8 |
| 4 | T*C_G*T*C_G*T*T*T_T*G*A*C_G*T*T*T*T*G*T*C_G*T*T | 24 | |
| 5 | T*C_G*T*C_G*T*T*T_T*G*A*C_G*T*T*T_T*G*T*C_G*T*T | 24 | |
| 6 | T*C_G*T*C_G*T_T*T_T*G_A*C_G*T_T*T T*G_T*C_G*T*T | 24 | |
| 7 | T_C_G_T_C_G_T_T_T_T_G_A_C_G_T_T_T_T_G_T_C_G_T_T | 24 | |
| 8 | G*T*C_G*T*T*T*T*G*A*C_G*T*T*T*T*G*T*C_G*T*T | 22 | 5′N-2 |
| 9 | T*C_G*T*C_G*T*T*T*T*G*A*C_G*T*T*T*T*G*T*C | 21 | 3′N-3 |
| 10 | T*C_G*T*C_G*T*T*T*T*G*A*C | 13 | 3′N-11 |
| 11 | G*T*T*T*T*G*A*C_G*T*T*T*T*G*T*C | 16 | 5′N-5, 3′N-3 |
| 12 | G*T*T*T*T*G*A*C_G*T*T*T*T*G*T*C_G*T*T | 19 | 5′N-5 |
| 13 | G*T*C_G*T*T*T*T*G*A*C_G*T*T | 14 | 5′N-2, 3′N-8 |
| 14 | T*C_G*T*T*T*T*G*A*C_G*T*T*T*T*G*T*C | 18 | 5′N-3, 3N-3 |
| 15 | G*T*C_G*T*T*T*T*G*A*C_G*T*T*T*T*G*T*C | 19 | 5′N-2, 3′N-3 |
| 16 | G*T*C_G*T*T*T*T*G*A*C | 11 | 5′N-2,3′N-11 |
| 17 | C_G*T*C_G*T*T*T*T*G*A*C_G*T*T*T*T*G*T*C_G*T*T | 23 | 5′N-I |
| 18 | T*C_I*T*C_I*T*T*T*T*G*A*C_I*T*T*T*T*G*T*C_I*T*T | 24 | CpG-CpI: |
| | | | Inosine (1) |
| 19 | T*MeC_G*T*MCC_G*T*T*T*T*G*A*MeC_G*T*T*T*T*G*T*MeC_G*T*T | 24 | CpG-MeCpG: |
| | | | 5-Methyl- |
| | | | Cytosine (MeC) |
| 20 | T*H_G*T*H_G*T*T*T*T*G*A*H G*T*T*T*T*G*T*H_G*T*T | 24 | CpG-HpG: 5- |
| | | | Hydroxy- |
| | | | Cytosine (H) |
| 21 | T*C_7*T*C_7*T*T*T*T*G*A*C_7*T*T*T*T*G*T*C_7*T*T | 24 | GpG-Cp7: 7- |
| | | | Deaza |
| | | | Guanosine (7) |
| 22 | U*C_G*U*C_G*U*U*U*U*G*A*C_G*U*U*U*U*G*U*C_G*U*U | 24 | T-U: Uracile (U) |

### Example 2

### Ability of Short Semi-soft CpG ODN to Activate TLR9

The same ODN tested in Example 1 were assayed in a TLR9 reporter gene system as described in Materials and Methods.

ODNs in different concentrations were tested in the TLR9 reporter gene assay. The EC50 was calculated using Sigma Plot (SigmaPlot 2002 for Windows Version 8.0). The maximal stimulation index (max SI) was calculated as the quotient between the highest value of all concentrations tested for any ODN and the medium control. The values are the mean of two independent experiments, with each data point determined in triplicate. The data is shown in Table 2.

TABLE 2
Stimulation index of TLR9 expressing cells by short semi-soft ODN.

| SEQ ID | EC50 [nM] | MAX SI |
|---|---|---|
| 1 | 240 | 49 |
| 2 | 955 | 17 |
| 3 | 5750 | 10 |
| 4 | 1245 | 15 |
| 5 | 3450 | 18 |
| 6 | 6200 | 12 |
| 7 | n/a | 1 |
| 8 | 945 | 18 |
| 9 | 1450 | 15 |
| 10 | 4800 | 10 |
| 11 | 3700 | 11 |
| 12 | 720 | 32 |
| 13 | 2150 | 43 |
| 14 | 625 | 50 |
| 15 | 480 | 46 |
| 16 | 4900/>5000 | 19 |
| 17 | 185 | 44 |
| 18 | 1550 | 18 |
| 19 | 935 | 10 |
| 20 | 1175 | 4 |
| 21 | 2050 | 3 |
| 22 | 6125 | 19 |

### Example 3

### Short ODN Semi-soft and Fully Hardened Demonstrate TLR9 Activity At Different Concentrations

HEK293 cells stably expressing human TLR9 and an NFκB-luciferase reporter construct were incubated for 16 h with the indicated ODN concentrations in the presence of DOTAP (N-[1-(2,3-Dioleoyloxy)propyl]-N,N,N-triethylammonium methylsulfate). Cells were lysed and TLR9 activation was determined by assaying luciferase activity. Simulation indices (SI) represent fold TLR9 activation in reference to activity of unstimulated cells. SI below 1.5 is considered to be background. The tested ODN and data are presented in Table 3.

TABLE 3

| SEQ ID | | | | |
|---|---|---|---|---|
| NO | Sequence 5′ - 3′ | Length | [μM] | SI TLR9 |
| 23 | T*G*T*C*G*T*T | 7 | 10 | 12.0 ± 1.2 |
| 23 | T*G*T*C*G*T*T | 7 | 25 | 17.6 ± 2.7 |
| 24 | T*G*T*C_G*T*T | 7 | 10 | 8.3 ± 1.1 |
| 24 | T*G*T*C_G*T*T | 7 | 25 | 18.4 ± 1.2 |
| 25 | G*T*C*G*T*T | 6 | 10 | 2.0 ± 0.1 |
| 25 | G*T*C*G*T*T | 6 | 25 | 8.4 ± 1.1 |
| 26 | G*T*C_G*T*T | 6 | 10 | 9.1 ± 1.4 |
| 26 | G*T*C_G*T*T | 6 | 25 | 25.7 ± 2.2 |
| 27 | G*T*C*G*T | 5 | 10 | 1.4 ± 0.1 |
| 27 | G*T*C*G*T | 5 | 25 | 2.1 ± 0.1 |
| 28 | G*T*C_G*T | 5 | 10 | 3.8 ± 0.6 |
| 28 | G*T*C_G*T | 5 | 25 | 4.8 ± 0.3 |
| 29 | T*C*G*T*T | 5 | 10 | 1.4 ± 0.06 |
| 29 | T*C*G*T*T | 5 | 25 | 2.1 ± 0.1 |
| 30 | T*C_G*T*T | 5 | 10 | 5.6 ± 0.2 |
| 30 | T*C_G*T*T | 5 | 25 | 6.2 ± 0.5 |
| 31 | C_G | 2 | 10 | 1.5 ± 0.1 |
| 31 | C_G | 2 | 25 | 1.6 ± 0.1 |

### Example 4

### Short Semi-soft and Fully Hardened ODNs Demonstrate IFN-alpha Induction At Different Concentrations

As shown in FIG. 2A and 2B, ODN SEQ ID NO.: 26 (a 7mer) and 24 (a 6mer), which are both semi-soft CpG containing ODNs, demonstrated strong IFN-alpha induction in the presence of DOTAP. The induction was stronger than that of the corresponding fully hardened CpG ODN SEQ ID NO.:25 and 23 (These ODNs the same sequence but lack a phosphodiester linkage between C and G). The same effect was detected with the shorter ODNs SEQ ID NO.:28 and 30 (containing a phosphodiester linkage) as compared to the hardened SEQ ID NO.:ODN 27 and 29. Induction of IFN-alpha above background was also seen with ODN SEQ ID NO.: 31.

### Example 5

### Ability Of Short ODNs with Modified Linkers to Activate TLR-9

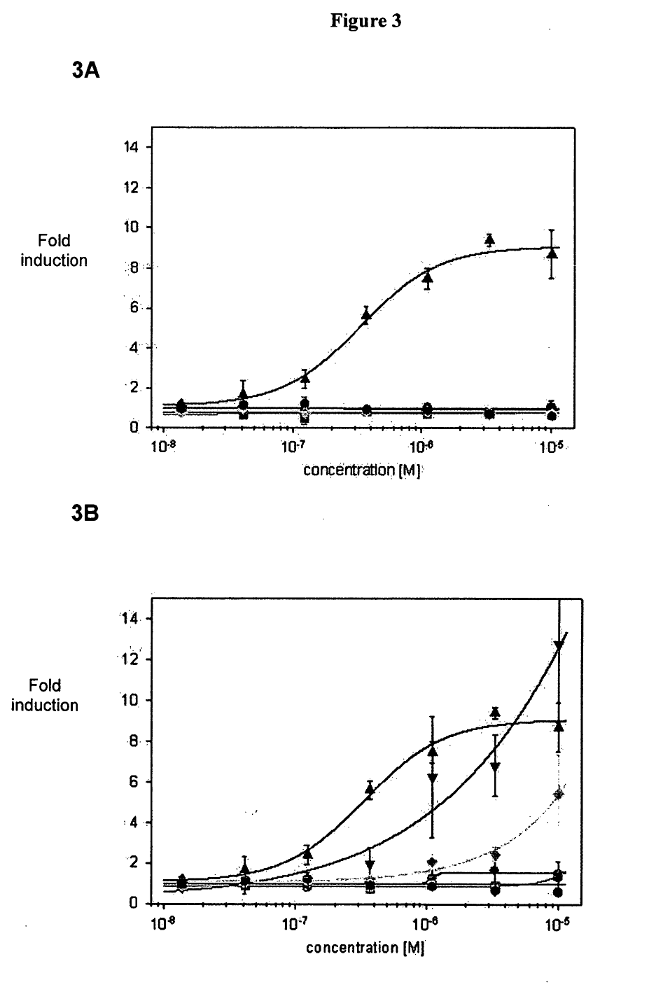

The ability of modified linkers to activate the TLR-9 receptor was investigated. Four ODNs with the same sequence but different linkers between the central C-G base pair were tested (ODN Sequences see Table 4). HEK293 cells stably expressing human TLR9 and an NFκB-luciferase reporter construct were incubated for 1 6 h with the different ODNs. Cells were lysed and TLR9 activation was determined by assaying luciferase activity. As can be seen in FIG. 3A, none of the short oligos were capable of activating TLR. The ODN 38, used as positive control, did show induction of TLR9.

To investigate the influence of a liposomal transfection agent on the TLR induction, the experiment was repeated by precomplexing the ODNs with DOTAP (N-[1-(2,3-Dioleoyloxy)propyl]-N,N,N-triethylammonium methylsulfate). The ratio of ODN to DOTAP was kept constant at 1 μM ODN to 10 μg/ml DOTAP. FIG. 3B shows that after complexing to DOTAP, the Semi-soft ODN (SEQ ID NO: 26) was capable of activating TLR.

### Example 6

### Ability Of Short ODNs with Modified Linkers to Induce Cytokine Expression in Human PBMC

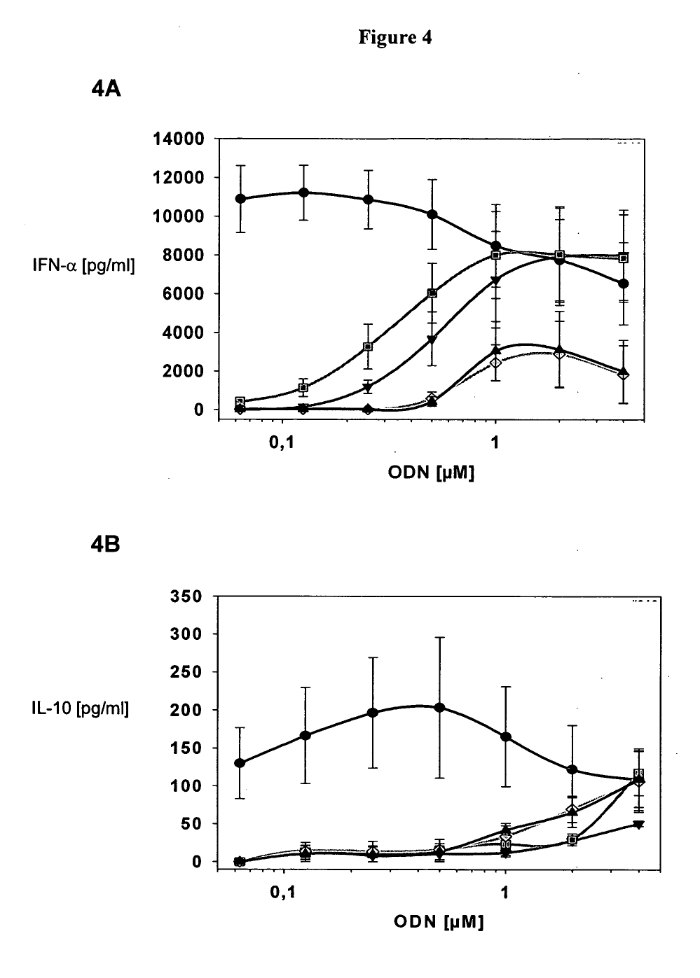

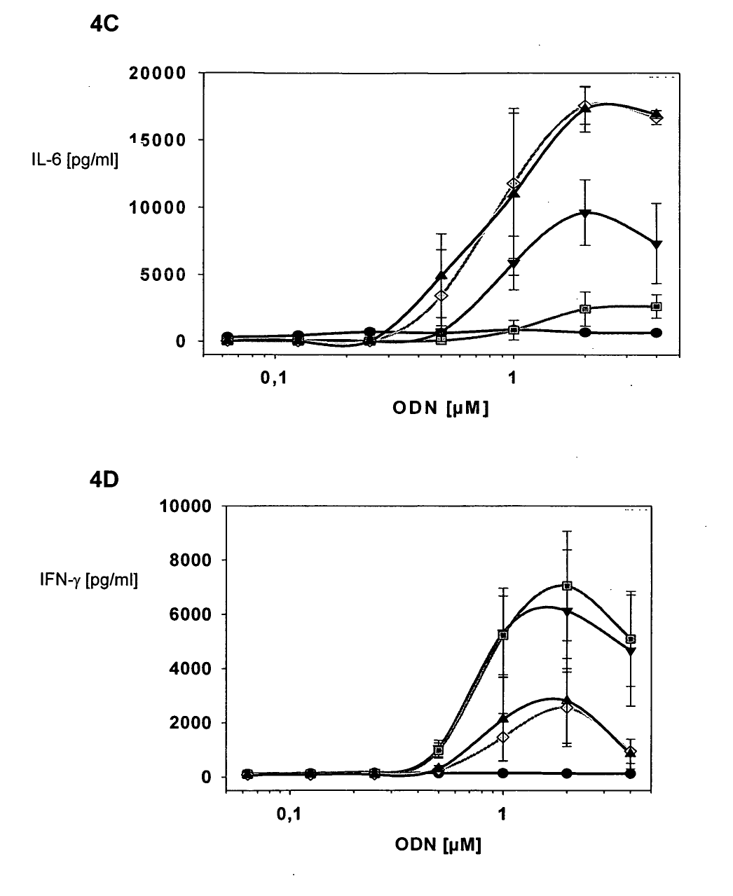

The same ODNs interrogated in Example 5, were tested for their ability to induce cytokine expression in PBMC. ODNs were precomplexed to DOTAP prior to addition to the cells. The ratio of ODN to DOTAP was kept constant at 1 μM ODN to 10 μg/ml DOTAP. As can be seen in FIG. 4A, the ODNs show a different ability to induce IFN-α secretion. The semisoft ODN (SEQ ID NO: 26) and the ODN with the unmodified linker showed the strongest induction profiles. The control ODN (SEQ ID NO: 38) was able to induce strong IFN-α secretion even at very low concentration. In the case of IL-10, the control ODN again induced secretion of the cytokine at all concentrations tested. None of the tested ODNs showed strong induction of IL-10 secretion (FIG. 4B).

When IL-6 secretion was monitored a different profile was observed. Strong induction was seen with the ODNs having methylphosphonate and ethylphosphate linkers (SEQ ID NO: 36 and SEQ ID NO: 37), while the unmodified linker showed a much lower response. Less induction was seen with the phosphorothiate ODN (SEQ ID NO: 25) and levels close to the control ODN (SEQ ID NO: 38) were observed for the semi-soft ODN (SEQ ID NO: 26)(FIG. 4C). Secretion of the IFN-γ cytokine showed a different picture yet again. Secretion was achived readily by exposure to the semisoft or unmodified ODNs. The ODNs with methylphosphonate or ethylphosphate linkers showed only moderate induction, while the control ODN (SEQ ID NO: 38) was not able to induce IFN-γ secretion (FIG. 4D).

### Example 7

### Ability Of Oligo Dinucleotides with Modified Linkers to Induce Cvtokine Expression In Human PBMC

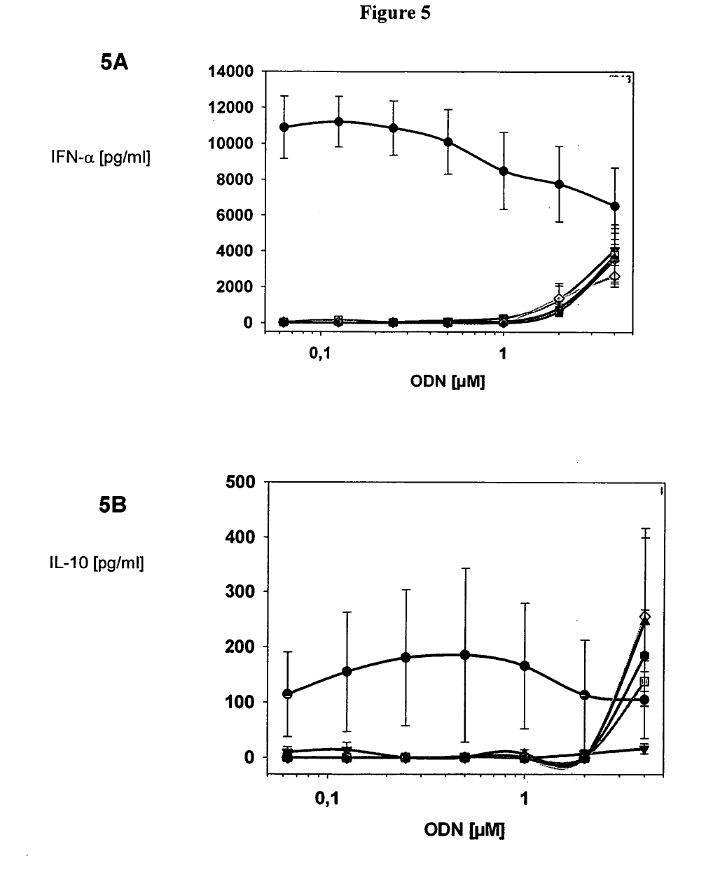

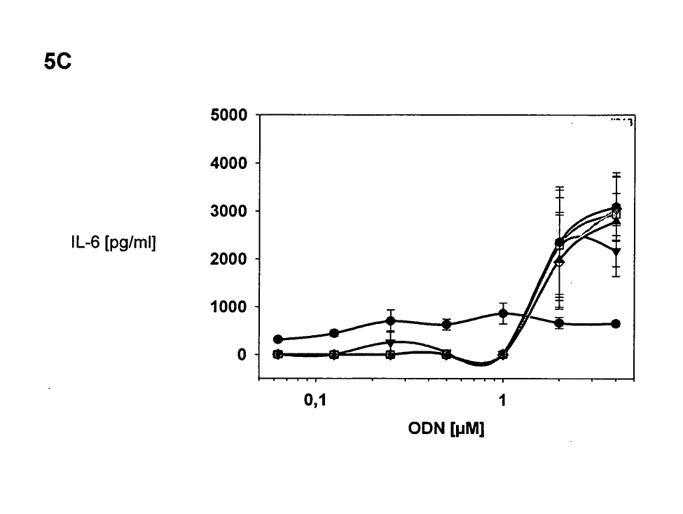

Five GC dinucleotides with different linkers were tested for their ability to induce cytokine secretion in PBMC. ODNs were precomplexed with DOTAP in a 1 μM ODN dioxolane to 10 μg/ml DOTAP ratio before they were added to the cells. The ODN dinucleotides were able to induce IFN-α secretion at high concentrations (FIG. 5A). As was seen in Example 6, the control ODN (SEQ ID NO: 38) was able to induce IFN-α secretion at all concentrations tested. Secretion of the cytokine IL-10 showed a similar induction profile. IL-10 could be induced by ODN (SEQ ID NO: 38) at all concentrations tested. Only at the highest concentration tested did the dimer ODNs show induction of IL-10 secretion. The 3′aminohexyl modified ODN (SEQ ID NO:40) did not demonstrate the ability to induce IL-10 secretion (FIG. 5B). When the secretion of the IL-6 cytokine was monitored a different pattern emerged. The control ODN (SEQ ID NO: 38) was capable of inducing moderate levels of IL-6 secretion at each of the concentrations tested. All dinucleotide ODNs tested were capable of inducing higher levels of IL-6 than induced by the control ODN (SEQ ID NO: 38), but only at higher concentrations (FIG. 5C).

### Example 8

### Ability Of the Double-dinucleotide (C-G-L)-2doub-but to Induce IFN-α Secretion In Human PBMC.

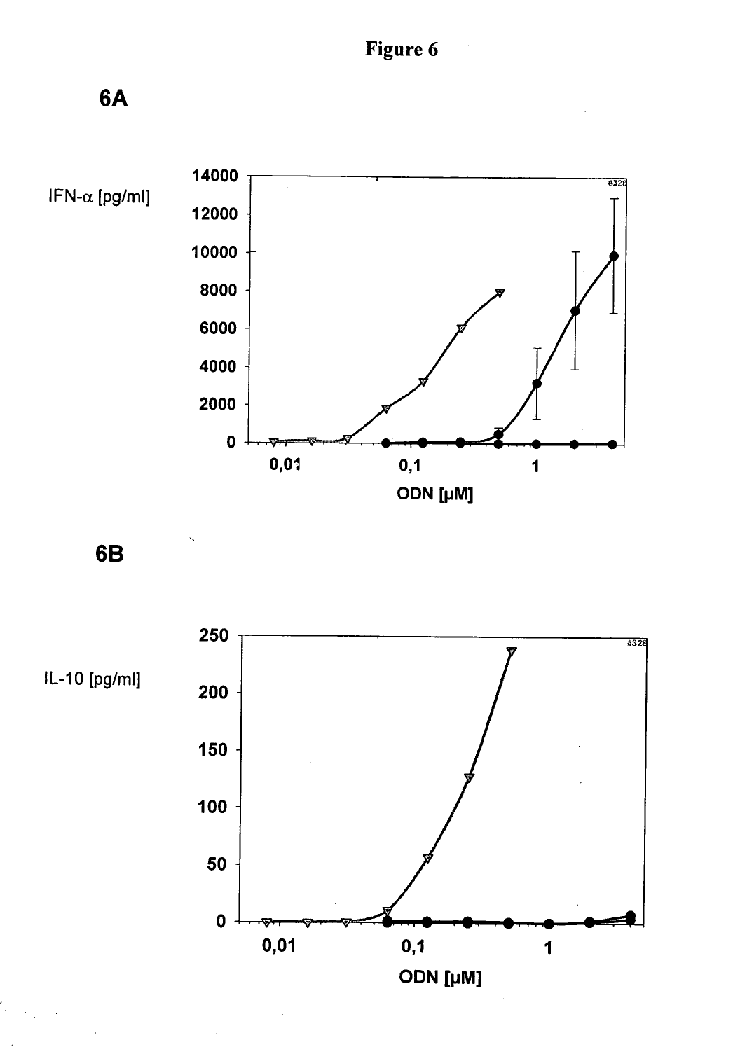

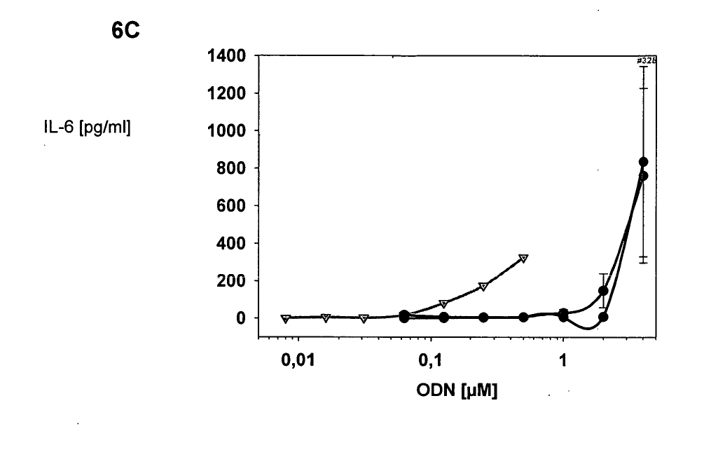

Levels of IFN-A secreted from human PBMC following exposure of these cells to the ODN (C-G-L)-2doub-but (SEQ ID NO: 43) and the positive control ODN (SEQ ID NO: 38) are shown in FIG. 6A. The concentration of the ODNs is depicted along the X-axis (μM). The ratio of ODN to DOTAP was kept constant at 4 μM ODN to 10 μg/ml DOTAP. The ODN was precomplexed with DOTAP before addition of the complex to PBMC. Both ODNs are capable of inducing IFN-α secretion, although ODN (SEQ ID NO: 38) is active at much lower concentrations.

As shown in FIG. 6B, the (C-G-L)-2doub-but ODN did not induce IL-10 secretion (In contrast to the control ODN). In the case of the IL-6 cytokine, the (C-G-L)-2doub-but did show induction of secretion at the higher concentration, but a negative control experiment with just DOTAP showed a similar induction (FIG. 6C).

TABLE 4
ODN sequences

| | | | |
|---|---|---|---|
| New | | | |
| Seq | | | |
| ID | ODN Sequence | length | comments |

| | | | |
|---|---|---|---|
| 1 | T*C_G*T*C_G*T*T*T*T*G*A*C_G*T*T*T*T*G*T*C_G*T*T | 24 | |
| 2 | T*C_G*T*T*T*T*G*A*C_G*T*T*T*T*G*T*C G*T*T | 21 | 5′N-3 |
| 3 | T*C_G*T*T*T*T*G*A*C_G*T*T | 13 | 5′N-3, 3′N-8 |
| 4 | T*C_G*T*C_G*T*T*T_T*G*A*C_G*T*T*T*T*G*T*C_G*T*T | 24 | |
| 5 | T*C_G*T*C_G*T*T*T_T*G*A*C_G*T*T*T_T*G*T*C_G*T*T | 24 | |
| 6 | T*C_G*T*C_G*T_T*T_T*G A*C_G*T_T*T_T*G_T*C_G*T*T | 24 | |
| 7 | T_C_G_T_C_G_T_T_T_T_G_A_C_G_T_T_T_T_G_T_C_G_T_T | 24 | |
| 8 | G*T*C_G*T*T*T*T*G*A*C_G*T*T*T*T*G*T*C_G*T*T | 22 | 5′N-2 |
| 9 | T*C_G*T*C_G*T*T*T*T*G*A*C_G*T*T*T*T*G*T*C | 21 | 3′N-3 |
| 10 | T*C_G*T*C_G*T*T*T*T*G*A*C | 13 | 3′N-11 |
| 11 | G*T*T*T*T*G*A*C_G*T*T*T*T*G*T*C | 16 | 5′N-5, 3′N-3 |
| 12 | G*T*T*T*T*G*A*C_G*T*T*T*T*G*T*C_G*T*T | 19 | 5′N-5 |
| 13 | G*T*C_G*T*T*T*T*G*A*C_G*T*T | 14 | 5′N-2, 3′N-8 |
| 14 | T*C_G*T*T*T*T*G*A*C_G*T*T*T*T*G*T*C | 18 | 5′N-3, 3′N-3 |
| 15 | G*T*C_G*T*T*T*T*G*A*C_G*T*T*T*T*G*T*C | 19 | 5′N-2, 3′N-3 |
| 16 | G*T*C_G*T*T*T*T*G*A*C | 11 | 5′N-2, 3′N-11 |
| 17 | C_G*T*C_G*T*T*T*T*G*A*C_G*T*T*T*T*G*T*C_G*T*T | 23 | 5′N-1 |
| 18 | T*C_I*T*C_I*T*T*T*T*G*A*C_I*T*T*T*T*G*T*C_I*T*T | 24 | CpG-CpI: |
| | | | Inosine (I) |
| 19 | T*MeC_G*T*MeC_G*T*T*T*T*G*A*MeC_G*T*T*T*T*G*T*MeC_G*T*T | 24 | CpG-MeCpG: |
| | | | 5′-Methyl- |
| | | | Cytosine |
| | | | (Mec) |
| 20 | T*H_G*T*H_G*T*T*T*T*G*A*H_G*T*T*T*T*G*T*H_G*T*T | 24 | CpG-HpG: 5- |
| | | | Hydroxy- |
| | | | Cytosine (H) |
| 21 | T*C_7*T*C_7*T*T*T*T*G*A*C_7*T*T*T*T*G*T*C_7*T*T | 24 | CpG-Cp7: 7- |
| | | | Deaza |
| | | | Guanosine (7) |
| 22 | U*C_G*U*C_G*U*U*U*U*G*A*C_G*U*U*U*U*G*U*C_G*U*U | 24 | T-U: Uracile |
| | | | (U) |
| 23 | T*G*T*C*G*T*T | 7 | |
| 24 | T*G*T*C_G*T*T | 7 | |
| 25 | G*T*C*G*T*T | 6 | |
| 26 | G*T*C_G*T*T | 6 | |
| 27 | G*T*C*G*T | 5 | |
| 28 | G*T*C_G*T | 5 | |
| 29 | T*C*G*T*T | 5 | |
| 30 | T*C_G*T*T | 5 | |
| 31 | C_G | 2 | |
| 32 | T*C_G*T*C_G*T*T*T*C_G*T*C_G*T*T | 16 | |
| 33 | T*C_G*T*C_G*T*T*T*T_G*T*C_G*T*T | 16 | |
| 34 | T*C*G*T*C*G*T*T*T*T*G*T*C*G*T*T*T*T*G*T*C*G*T*T | 24 | |
| 35 | T*C_G*T*C_G*T*T*T*T_G*T*C_G*T*T*T*T*G*T*G_G*T*T | 24 | |
| 36 | G*T*C§G*T*T | 6 | methyl- |
| | | | phosphonate |
| 37 | G*T*C+G*T*T | 6 | ethylphosphate |
| 38 | T*C*G*T*C*G*T*T*T*T*T*C*G*G*T*C*G*T*T*T*T | 21 | |
| 39 | C*G | 2 | |
| 40 | C-G-iami-6 | 2 | 3′aminohexyl |
| 41 | ami6-C-G | 2 | 5′aminohexyl |
| 42 | ami6-C-G-iami6 | 2 | 3′5′bis |
| | | | aminohexyl |
| 43 | (C-G-L-)2doub-but | 2 × 2 | hexaethylenegl |
| | | | ycol linkers |
| | | | doubler |
| | | | phosphoroami |
| | | | dite |
| | | | butyrate |

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

We claim:

1. An oligonucleotide of 3 to 24 nucleotides in length comprising at least one YZ dinucleotide with a phosphodiester or phosphodiester-like internucleotide linkage, and at least 4 T nucleotides, wherein Y is a nucleotide comprising a pyrimidine or modified pyrimidine base, wherein Z is a nucleotide comprising a guanine or modified guanine, and wherein the oligonucleotide includes at least one stabilized internucleotide linkage.

2. The oligonucleotide of claim 1, wherein the oligonucleotide includes a TTTT motif.

3. The oligonucleotide of claim 2, wherein the oligonucleotide has only one YZ dinucleotide.

4. The oligonucleotide of claim 3, wherein the oligonucleotide is G*T*C_G*T*T*T*T*G*A*C (SEQ ID NO.: 16) or G*T*T*T*T*G*A*C_G*T*T*T*T*G*T*C (SEQ ID NO.: 11), wherein *refers to the presence of a stabilized internucleotide linkage, and wherein _refers to the presence of a phosphodiester internucleotide linkage.

5. The oligonucleotide of claim 2, wherein the oligonucleotide has only two YZ dinucleotides.

6. The oligonucleotide of claim 5, wherein the oligonucleotide is selected from the group consisting of T*C_G*T*T*T*T*G*A*C_G*T*T (SEQ ID NO.: 3), T*C_G*T*C_G*T*T*T*T*G*A*C (SEQ ID NO.: 10), G*T*T*T*T*G*A*C_G*T*T*T*T*G*T*C_G*T*T (SEQ ID NO.: 12), G*T*C_G*T*T*T*T*G*A*C_G*T*T (SEQ ID NO.: 13), T*C_G*T*T*T*T*G*A*C_G*T*T*T*T*G*T*C (SEQ ID NO.: 14), and G*T*C_G*T*T*T*T*G*A*C_G*T*T*T*T*G*T*C (SEQ ID NO.: 15), wherein *refers to the presence of a stabilized internucleotide linkage, and wherein _ refers to the presence of a phosphodiester internucleotide linkage.

7. The oligonucleotide of claim 2, wherein the oligonucleotide has only three YZ dinucleotides.

8. The oligonucleotide of claim 7, wherein the oligonucleotide is selected from the group consisting of T*C_G*T*T*T*T*G*A*C_G*T*T*T*T*G*T*C_G*T*T (SEQ ID NO.: 2), G*T*C_G*T*T*T*T*G*A*C_G*T*T*T*T*G*T*C_G*T*T (SEQ ID NO.: 8), T*C_G*T*C_G*T*T*T*T*G*A*C_G*T*T*T*T*G*T*C (SEQ ID NO.: 9), and T*C_G*T*C_G*T*T*T*T*G*A*C (SEQ ID NO.: 10), wherein *refers to the presence of a stabilized internucleotide linkage, and wherein _refers to the presence of a phosphodiester internucleotide linkage.

9. The oligonucleotide of claim 2, wherein the oligonucleotide has only four YZ dinucleotides.

10. The oligonucleotide of claim 9, wherein the oligonucleotide is selected from the group consisting of T*C_G*T*C_G*T*T*T_T*G*A*C_G*T*T*T*T*G*T*C_G*T*T (SEQ ID NO.: 4), T*C_G*T*C_G*T*T*T_T*G*A*C_G*T*T*T₁₃ T*G*T*C_G*T*T (SEQ ID NO.:5), T*C_G*T*C_G*T_T*T_T*G_A*C_G*T_T*T_T*G_T*C_G*T*T (SEQ ID NO.: 6), C_G*T*C_G*T*T*T*T*G*A*C_G*T*T*T*T*G*T*C_G*T*T (SEQ ID NO.: 17), T*C_I*T*C_I*T*T*T*T*G*A*C_I*T*T*T*T*G*T*C_I*T*T (SEQ ID NO.: 18), T* MeC _G*T* MeC _G*T*T*T*T*G*A*MeC_G*T*T*T*T*G*T*MeC_G*T*T (SEQ ID NO.: 19), T*H_G*T*H_G*T*T*T*T*G*A*H_G*T*T*T*T*G*T*H_G*T*T (SEQ ID NO.: 20), T*C_{—}7*T*C_{—}7*T*T*T*T*G*A*C_{—}7*T*T*T*T*G*T*C_{—}7*T*T (SEQ ID NO.: 21), and U*C_G*U*C_G*U*U*U*U*G*A*C_G*U*U*U*U*G*U*C_G*U*U (SEQ ID NO.: 22), wherein *refers to the presence of a stabilized internucleotide linkage, wherein _refers to the presence of a phosphodiester internucleotide linkage, and wherein I is Inosine comprising a Hypoxanthine base; MeC is 5′-Methyl-Cytosine, H is 5-Hydroxy-Cytosine, 7 is 7-Deaza-Guanine, and U is Uracil.

11. The oligonucleotide of claim 1, wherein each YZ dinucleotide has a phosphodiester or phosphodiester-like internucleotide linkage.

12. The oligonucleotide of claim 1, wherein Y is a nucleotide comprising an unmethylated cytosine.

13. The oligonucleotide of claim 1, wherein Z is a nucleotide comprising a guanine.

14. The oligonucleotide of claim 1, wherein the phosphodiester-like linkage is boranophosphonate or diastereomerically pure Rp phosphorothioate.

15. The oligonucleotide of claim 1, wherein the stabilized internucleotide linkages are selected from the group consisting of: phosphorothioate, phosphorodithioate, methylphosphonate, methylphosphorothioate, and any combination thereof.

16. The oligonucleotide of claim 1, wherein Y is a nucleotide comprising a cytosine or a modified cystosine base selected from the group consisting of 5-methyl cytosine, 5-methyl-isocytosine, 5-hydroxy-cytosine, 5-halogeno cytosine, uracil, N4-ethyl-cytosine, 5-fluoro-uracil, and hydrogen.

17. The oligonucleotide of claim 1, wherein Z is a nucleotide comprising a guanine or a modified guanine base selected from the group consisting of 7-deazaguanine, 7-deaza-7-substituted guanine (such as 7-deaza-7-(C2-C6)alkynylguanine), 7-deaza-8-substituted guanine, hypoxanthine, 2,6-diaminopurine, 2-aminopurine, purine, 8-substituted guanine such as 8-hydroxyguanine, and 6-thioguanine, 2-aminopurine, and hydrogen.

18. The oligonucleotide of claim 1, wherein the oligonucleotide has a 3′-3′ linkage with one or two accessible 5′ ends.

19. The oligonucleotide of claim 1, wherein the oligonucleotide has two accessible 5′ ends, each of which are 5′TCG.

20. An oligonucleotide of 2 to 7 nucleotides in length, wherein the oligonucleotide has at least one YZ dinucleotide with a phosphodiester or phosphodiester-like internucleotide linkage, wherein Y is a a nucleotide comprising a pyrimidine or modified pyrimidine base, wherein Z is a nucleotide comprising a guanine or modified guanine, and wherein the oligonucleotide includes at least one stabilized internucleotide linkage.

21. The oligonucleotide of claim 20, wherein the oligonucleotide has only one YZ dinucleotide.

22. The oligonucleotide of claim 20, wherein the oligonucleotide is selected from the group consisting of T*G*T*C*G*T*T (SEQ ID NO.: 23), T*G*T*C_G*T*T (SEQ ID NO.: 24), G*T*C*G*T*T (SEQ ID NO.: 25), G*T*C_G*T*T (SEQ ID NO.: 26), G*T*C*G*T (SEQ ID NO.: 27), G*T*C_G*T (SEQ ID NO.: 28), T*C*G*T*T (SEQ ID NO.: 29), T*C_G*T*T (SEQ ID NO.: 30), and C_G (SEQ ID NO.: 31), wherein * refers to the presence of a stabilized internucleotide linkage, and wherein _refers to the presence of a phosphodiester internucleotide linkage.

23. The oligonucleotide of claim 20, wherein Y is an unmethylated C.

24. The oligonucleotide of claim 20, wherein Z is a nucleotide comprising a guanine.

25. The oligonucleotide of claim 20, wherein the stabilized internucleotide linkage is phosphorothioate.

26. The oligonucleotide of claim 20, wherein Y is a nucleotide comprising a cytosine or a modified cystosine base selected from the group consisting of 5-methyl cytosine, 5-methyl-isocytosine, 5-hydroxy-cytosine, 5-halogeno cytosine, uracil, N4-ethyl-cytosine, 5-fluoro-uracil, and hydrogen.

27. The oligonucleotide of claim 20, wherein Z is a nucleotide comprising a guanine or a modified guanine base selected from the group consisting of 7-deazaguanine, 7-deaza-7-substituted guanine (such as 7-deaza-7-(C2-C6)alkynylguanine), 7-deaza-8-substituted guanine, hypoxanthine, 2,6-diaminopurine, 2-aminopurine, purine, 8-substituted guanine such as 8-hydroxyguanine, and 6-thioguanine, 2-aminopurine, and hydrogen.

28. The oligonucleotide of claim 20, wherein the oligonucleotide has a 3′-3′ linkage with one or two accessible 5′ ends.

29. The oligonucleotide of claim 20, wherein the oligonucleotide has two accessible 5′ ends, each of which are 5′TCG.

30. An oligonucleotide of 7 nucleotides in length, wherein the oligonucleotide has at least one CG dinucleotide, wherein the oligonucleotide includes at least one stabilized internucleotide linkage.

31. The oligonucleotide of claim 30, wherein the all of the internucleotide linkages are phosphorothioate linkages.

32. An oligonucleotide of 5 to 7 nucleotides in length, wherein the oligonucleotide comprises GTCGT or TCGTT, and wherein the oligonucleotide includes at least one stabilized internucleotide linkage.

33. The oligonucleotide of claim 32, wherein the all of the internucleotide linkages are phosphorothioate linkages.

34. An oligonucleotide comprising at least one YZ dinucleotide with a linkage that is an ethylphosphate or methylphosphonate, wherein Y is a nucleotide comprising a pyrimidine or modified pyrimidine base, wherein Z is a nucleotide comprising a guanine or modified guanine.

35. The oligonucleotide of claim 34, wherein the oligonucleotide has a length of 4-100 nucleotides.

36. An oligonucleotide comprising at least one YZ dinucleotide with a phosphodiester or phosphodiester-like internucleotide linkage, and wherein Y is a nucleotide comprising a pyrimidine or modified pyrimidine base, wherein Z is a nucleotide comprising a guanine or modified guanine, and wherein the oligonucleotide contains an aminohexylgroup at the 3′ end of the oligonucleotide.

37. An oligonucleotide comprising at least one YZ dinucleotide with a phosphodiester or phosphodiester-like internucleotide linkage, and wherein Y is a nucleotide comprising a pyrimidine or modified pyrimidine base, wherein Z is a nucleotide comprising a guanine or modified guanine, and wherein the oligonucleotide contains an aminohexylgroup at the 5′ end of the oligonucleotide.

38. An oligonucleotide comprising at least one YZ dinucleotide with a phosphodiester or phosphodiester-like internucleotide linkage, and wherein Y is a nucleotide comprising a pyrimidine or modified pyrimidine base, wherein Z is a nucleotide comprising a guanine or modified guanine, and wherein the oligonucleotide contains an aminohexylgroup at the 5′ and 3′ ends of the oligonucleotide.

39. The oligonucleotide of claim 38, wherein the oligonucleotide includes at least one stabilized internucleotide linkage.

40. The oligonucleotide of claim 38, wherein the oligonucleotide has a length of 4-100 nucleotides.

41. The oligonucleotide of claim 38, wherein Y is a nucleotide comprising an unmethylated cytosine.

42. A method for treating cancer, comprising administering an oligonucleotide of claim 1, to a subject having cancer in an effective amount to treat the cancer.

43. A method for treating allergy, comprising administering an oligonucleotide of claim 41, to a subject having or at risk of having an allergy in an effective amount to treat the allergy.

44. A method for treating asthma, comprising administering an oligonucleotide of claim 1, to a subject having asthma in an effective amount to treat the asthma.

45. A method for treating infectious disease, comprising administering an oligonucleotide of claim 1, to a subject having or at risk of having infectious disease in an effective amount to treat the infectious disease.


SEQUENCE LISTING (43 sequences)

SEQ ID NO: 1 - DNA, 24 bp, Artificial Sequence
  Synthetic oligonucleotide
tcgtcgttttgacgttttgtcgtt

SEQ ID NO: 2 - DNA, 21 bp, Artificial Sequence
  Synthetic oligonucleotide
tcgttttgacgttttgtcgtt

SEQ ID NO: 3 - DNA, 13 bp, Artificial Sequence
  Synthetic oligonucleotide
tcgttttgacgtt

SEQ ID NO: 4 - DNA, 24 bp, Artificial Sequence
  Synthetic oligonucleotide
tcgtcgttttgacgttttgtcgtt

SEQ ID NO: 5 - DNA, 24 bp, Artificial Sequence
  Synthetic oligonucleotide
tcgtcgttttgacgttttgtcgtt

SEQ ID NO: 6 - DNA, 24 bp, Artificial Sequence
  Synthetic oligonucleotide
tcgtcgttttgacgttttgtcgtt

SEQ ID NO: 7 - DNA, 24 bp, Artificial Sequence
  Synthetic oligonucleotide
tcgtcgttttgacgttttgtcgtt

SEQ ID NO: 8 - DNA, 22 bp, Artificial Sequence
  Synthetic oligonucleotide
gtcgttttgacgttttgtcgtt

SEQ ID NO: 9 - DNA, 21 bp, Artificial Sequence
  Synthetic oligonucleotide
tcgtcgttttgacgttttgtc

SEQ ID NO: 10 - DNA, 13 bp, Artificial Sequence
  Synthetic oligonucleotide
tcgtcgttttgac

SEQ ID NO: 11 - DNA, 16 bp, Artificial Sequence
  Synthetic oligonucleotide
gttttgacgttttgtc

SEQ ID NO: 12 - DNA, 19 bp, Artificial Sequence
  Synthetic oligonucleotide
gttttgacgttttgtcgtt

SEQ ID NO: 13 - DNA, 14 bp, Artificial Sequence
  Synthetic oligonucleotide
gtcgttttgacgtt

SEQ ID NO: 14 - DNA, 18 bp, Artificial Sequence
  Synthetic oligonucleotide
tcgttttgacgttttgtc

SEQ ID NO: 15 - DNA, 19 bp, Artificial Sequence
  Synthetic oligonucleotide
gtcgttttgacgttttgtc

SEQ ID NO: 16 - DNA, 11 bp, Artificial Sequence
  Synthetic oligonucleotide
gtcgttttgac

SEQ ID NO: 17 - DNA, 23 bp, Artificial Sequence
  Synthetic oligonucleotide
cgtcgttttgacgttttgtcgtt

SEQ ID NO: 18 - DNA, 24 bp, Artificial Sequence
  Synthetic oligonucleotide
tcntcnttttgacnttttgtcntt

SEQ ID NO: 19 - DNA, 24 bp, Artificial Sequence
  Synthetic oligonucleotide
tcgtcgttttgacgttttgtcgtt

SEQ ID NO: 20 - DNA, 24 bp, Artificial Sequence
  Synthetic oligonucleotide
tngtngttttgangttttgtngtt

SEQ ID NO: 21 - DNA, 24 bp, Artificial Sequence
  Synthetic oligonucleotide
tcntcnttttgacnttttgtcntt

SEQ ID NO: 22 - DNA, 24 bp, Artificial Sequence
  Synthetic oligonucleotide
ncgncgnnnngacgnnnngncgnn

SEQ ID NO: 23 - DNA, 7 bp, Artificial Sequence
  Synthetic oligonucleotide
tgtcgtt

SEQ ID NO: 24 - DNA, 7 bp, Artificial Sequence
  Synthetic oligonucleotide
tgtcgtt

SEQ ID NO: 25 - DNA, 6 bp, Artificial Sequence
  Synthetic oligonucleotide
gtcgtt

SEQ ID NO: 26 - DNA, 6 bp, Artificial Sequence
  Synthetic oligonucleotide
gtcgtt

SEQ ID NO: 27 - DNA, 5 bp, Artificial Sequence
  Synthetic oligonucleotide
gtcgt

SEQ ID NO: 28 - DNA, 5 bp, Artificial Sequence
  Synthetic oligonucleotide
gtcgt

SEQ ID NO: 29 - DNA, 5 bp, Artificial Sequence
  Synthetic oligonucleotide
tcgtt

SEQ ID NO: 30 - DNA, 5 bp, Artificial Sequence
  Synthetic oligonucleotide
tcgtt

SEQ ID NO: 31 - DNA, 2 bp, Artificial Sequence
  Synthetic oligonucleotide
cg

SEQ ID NO: 32 - DNA, 16 bp, Artificial Sequence
  Synthetic oligonucleotide
tcgtcgtttcgtcgtt

SEQ ID NO: 33 - DNA, 16 bp, Artificial Sequence
  Synthetic oligonucleotide
tcgtcgttttgtcgtt

SEQ ID NO: 34 - DNA, 24 bp, Artificial Sequence
  Synthetic oligonucleotide
tcgtcgttttgtcgttttgtcgtt

SEQ ID NO: 35 - DNA, 24 bp, Artificial Sequence
  Synthetic oligonucleotide
tcgtcgttttgtcgttttgtcgtt

SEQ ID NO: 36 - DNA, 6 bp, Artificial Sequence
  Synthetic oligonucleotide
gtcgtt

SEQ ID NO: 37 - DNA, 6 bp, Artificial Sequence
  Synthetic oligonucleotide
gtcgtt

SEQ ID NO: 38 - DNA, 21 bp, Artificial Sequence
  Synthetic oligonucleotide
tcgtcgtttttcggtcgtttt

SEQ ID NO: 39 - DNA, 2 bp, Artificial Sequence
  Synthetic oligonucleotide
cg

SEQ ID NO: 40 - DNA, 2 bp, Artificial Sequence
  Synthetic oligonucleotide
cg

SEQ ID NO: 41 - DNA, 2 bp, Artificial Sequence
  Synthetic oligonucleotide
cg

SEQ ID NO: 42 - DNA, 2 bp, Artificial Sequence
  Synthetic oligonucleotide
cg

SEQ ID NO: 43 - DNA, 4 bp, Artificial Sequence
  Synthetic oligonucleotide
cggc